(19) United States
(12) Patent Application Publication
(10) Pub. No.: US 2021/0196989 A1
(43) Pub. Date: Jul. 1, 2021

(54) Implantable Bio-Heating System Based on Piezoelectric Micromachined Ultrasonic Transducers

(71) Applicant: Northeastern University, Boston, MA (US)

(72) Inventors: Matteo RINALDI, Boston, MA (US); Flavius POP, Boston, MA (US); Bernard HERRERA, Cambridge, MA (US)

(21) Appl. No.: 17/121,703

(22) Filed: Dec. 14, 2020

Related U.S. Application Data
(60) Provisional application No. 62/947,654, filed on Dec. 13, 2019.

(51) Int. Cl. A61N 7/02; B06B 1/02; B06B 1/06

(52) U.S. Cl. CPC A61N 7/02; B06B 1/0215; B06B 1/0622; H01L 41/18; B06B 2201/55; B06B 2201/76; B06B 2201/20; A61N 7/022

(57) ABSTRACT

Implantable bio-heating and intrabody communication systems use arrays of piezoelectric micromachined ultrasonic transducers (pMUTs) to provide ultrasound-based diagnosis and treatment of medical conditions. Systems involving one or more pMUT arrays can be implanted into the body or integrating into smart ingestible pills to enable monitoring of a medical condition and/or continuous or intermittent application of hyperthermia and other treatments.

### CROSS-REFERENCE TO RELATED APPLICATIONS

This application claims priority to U.S. Provisional Application No. 62/947,654, filed 13 Dec. 2019, which is incorporated by reference herein in its entirety.

### STATEMENT REGARDING FEDERALLY SPONSORED RESEARCH OR DEVELOPMENT

This invention was made with government support under Grant Numbers 1618731 and 1726512 awarded by the National Science Foundation. The government has certain rights in the invention.

### BACKGROUND

Hyperthermia treatment (also called thermal therapy or thermotherapy) is a treatment in which body tissue is exposed to high temperatures to treat various diseases and infections. High temperatures can damage and kill cancer cells and some infectious agents. Damage to normal tissues can also occur through exposure to high temperatures used in hyperthermia treatment. Hyperthermia can also be used to enhance the effects of certain anticancer drugs and other treatments.

The effectiveness of hyperthermia treatment is related to a number of factors that can include the temperature achieved during the treatment, the duration of the treatment, the localization of the treatment, the cells and tissue treated, and the disease characteristics. To ensure that the desired temperature is reached, but not exceeded, the temperature of the treated area and surrounding tissue can be monitored throughout hyperthermia treatment. Improved techniques are needed for treating diseases with hyperthermia.

### SUMMARY

The technology described herein provides implantable bio-heating systems as well as outside the body implanted device scanners based on piezoelectric micromachined ultrasonic transducers (pMUTs). Given the biocompatibly and potential for miniaturization of pMUTs, the technology makes possible implantable devices and systems for ultrasonic therapies and ultrasonic communications with implanted medical devices.

In systems of the present technology, pMUTs are fabricated in arrays which are capable of focusing ultrasound for use in ultrasound therapy, hyperthermia treatment, targeted tissue ablation, or targeted communications with implanted medical devices. The arrays can be implanted in desired locations in the body of a patient or configured as smart ingestible pills, for monitoring of medical conditions or continuous and non-invasive application of the ultrasound therapy with post-treatment monitoring of the effects on a patient.

For hyperthermia treatment, a single 5×10 pMUT array can produce a 4° C. increase in temperature of a targeted aqueous medium in less than 10 seconds, allowing local heating of tissue from 37° C. to 41° C. The technology also includes systems and methods that combine several pMUT arrays to increase the heating capacity and/or focused delivery of ultrasound energy.

The present technology can be further summarized by the following list of features.

1. A system for ultrasonically heating biological tissue, comprising:

an implantable or ingestible device comprising:
a substrate, and
an array of piezoelectric micromachined ultrasonic transducers supported on the substrate, the substrate and the array implantable or ingestible in a body;

wherein the array is in communication with a controller operative to control the array to focus ultrasonic transmissions at biological tissue in the body to heat the biological tissue.

2. The system of feature 1, wherein the array of piezoelectric micromachined ultrasonic transducers comprises a piezoelectric layer activatable by application of a voltage to a pair of electrodes, the piezoelectric layer comprising aluminum nitride, scandium doped aluminum nitride, lithium niobate, or a combination thereof.
3. The system of feature 1, wherein each of the piezoelectric micromachined ultrasonic transducers is in communication with a controller operative to change the focal point of the array by providing a time delay to an AC voltage applied to each of the piezoelectric micromachined ultrasonic transducers.
4. The system of feature 1, wherein two or more arrays are provided and each array is configured with the piezoelectric micromachined ultrasonic transducers connected in a parallel configuration.
5. The system of feature 4, wherein each of the two or more arrays is in communication with a controller operative to change the focal point of each array by providing a time delay to the AC voltage applied to each array.
6. The system of feature 1, wherein the controller comprises a plurality of directly modulated ultrasonic transducer circuits, each circuit comprising an inductor, a capacitor, a voltage input, and a bipolar junction transistor.
7. The system of feature 6, further comprising a microprocessor in communication with the controller and operative to provide an input voltage to each of the plurality of directly modulated ultrasonic transducer circuits.
8. The system of feature 1, wherein the controller is implantable or ingestible.
9. The system of feature 1, wherein the piezoelectric micromachined ultrasonic transducers have a frequency response in a band in the range from about 300 kHz to about 10 MHz or in a band in the range from about 300 kHz to about 100 MHz.
10. The system of feature 1, wherein the system is operative to heat biological tissue to at least about 41° C. or to at least about 60° C.
11. An implantable, ingestible, or wearable medical device comprising the system of feature 1.
12. A method of ultrasonically heating biological tissue in a subject comprising:

(a) implanting or ingesting a device comprising:
a substrate, and
an array of piezoelectric micromachined ultrasonic transducers supported on the substrate, the substrate and the array implantable or ingestible in a body, wherein the array is in communication with a controller operative to control the array to focus ultrasonic transmissions at biological tissue in the body to heat the biological tissue; and

(b) heating biological tissue in the subject at a focal point of the device.

13. The method of feature 12, wherein each of the piezoelectric micromachined ultrasonic transducers is in communication with a controller operative to change the focal point of the array by providing a time delay to an AC voltage applied to each of the piezoelectric micromachined ultrasonic transducers.
14. The method of feature 12, wherein two or more arrays are provided and each array is configured with the piezoelectric micromachined ultrasonic transducers connected in a parallel configuration.
15. The method of feature 13, wherein each of the two or more arrays is in communication with a controller operative to change the focal point of each array by providing a time delay to the AC voltage applied to each array.
16. The method of feature 12, wherein the biological tissue is heated to at least about 41° C. or to at least about 60° C.
17. A method of heating cancer cells located in a biological tissue, the method comprising the method of feature 12, wherein the method is utilized to heat biological tissue comprising neck cancer cells, brain cancer cells, thyroid cancer cells, breast cancer cells, prostate cancer cells, kidney cancer cells, endometrial cancer cells, pancreatic cancer cells, lung cancer cells, esophageal cancer cells, bladder cancer cells, rectal cancer cells, cervical cancer cells, ovarian cancer cells, peritoneal cancer cells, sarcoma cancer cells, neuroblastoma cancer cells, leukemia cancer cells, melanoma cancer cells, or a combination thereof.
18. The method of feature 12, further comprising providing an imaging system operative to locate cancer cells; the imaging system comprising memory, software, and a processor in communication with a controller operative to change the focal point of the array; and (c) locating cancer cells, changing the focal point of the device to the location, and repeating step (b).
19. The method of feature 12, wherein the method is utilized in a combination with one or more of radiation therapy, immunotherapy, targeted drug therapy, chemotherapy, radiofrequency therapy, tumor imaging, and hormone therapy.
20. The method of feature 12, wherein the biological tissue is heated to about 41° C. within a time of less than about 10 seconds.
21. A method of treating cancer in a subject comprising the method of feature 12.
22. A method of tissue ablation comprising the method of feature 12.
23. An implantable or ingestible device comprising:
one or more substrates;
one or more arrays of piezoelectric micromachined ultrasonic transducers comprising a piezoelectric layer activatable by application of a voltage to a pair of electrodes, each of the one or more arrays supported on the one or more substrates, and each of the one or more arrays disposed at a distance from each other of the one or more arrays; and
a controller comprising a power supply and an electrical circuit in connection with each of the one or more arrays,
wherein the one or more substrates, the one or more arrays, and the controller are implantable or ingestible in a living subject.
   24. The device of feature 23 configured as an ingestible pill.
   25. The device of feature 23, wherein the one or more substrates are disposed on and/or in an implantable or ingestible support.
   26. The device of feature 23, further comprising a microprocessor in connection with the controller.
   27. The device of feature 23, wherein the power supply includes an ultrasonic transducer comprising an array of piezoelectric micromachined ultrasonic transducers operative to receive ultrasound and to convert ultrasound to electrical energy.
   28. The device of feature 23, wherein the controller includes an ultrasonic transceiver comprising an array of piezoelectric micromachined ultrasonic transducers and operative to decode an ultrasonic signal, in connection with a processing unit including memory and a processor operative to provide an input to the electrical circuit.
   29. The device of feature 23, further comprising an array of piezoelectric micromachined ultrasonic transducers operative to receive an ultrasonic signal at each of the piezoelectric micromachined ultrasonic transducers and to transduce a voltage from the ultrasonic signal at a pair of electrodes.
   30. The device of feature 23, wherein the piezoelectric micromachined ultrasonic transducers have a frequency response in a band in the range from about 300 kHz to about 10 MHz or in the range from about 300 kHz to about 100 MHz.
   31. A method of treating a disease or a condition in a subject, the method comprising:

implanting or ingesting the device of feature 23 in the subject; and

heating biological tissue in the subject at a focal point of the device.

32. The method of feature 31, further comprising measuring the temperature of the biological tissue in the subject.
33. The method of feature 31, further comprising implanting or ingesting an active agent in the subject.
34. The method of feature 32, wherein the active agent comprises a chemotherapy agent, a radioactive agent, nanoparticles, an imaging agent, a pharmaceutical agent, a biomolecule agent, or a combination thereof.
34. The method of feature 31, wherein the biological tissue is heated to a temperature of greater than about 40° C. after a time of less than about 10 seconds.

35. The method of feature 31, wherein the biological tissue is heated to a temperature of greater than about 50° C. after a time of less than about 10 seconds.

36. The method of feature 31, wherein the device of feature 23 is left in the subject for a time period of greater than about 24 hours, greater than about one week, greater than about one month, or greater than about one year.
37. The method of feature 31 wherein the disease or condition comprises head or neck cancer, brain cancer, thyroid cancer, breast cancer, prostate cancer, kidney cancer, endometrial cancer, pancreatic cancer, lung cancer, esophageal cancer, bladder cancer, rectal cancer, cervical cancer, ovarian cancer, peritoneal cancer, sarcoma cancer, neuroblastoma, leukemia, melanoma, a microbial infection, a viral infection, a heart or an organ condition, or a combination thereof.
38. The method of feature 31, further comprising monitoring the biological tissue for a formation of bubbles or a cavitation.
39. A method of monitoring a condition in a subject, the method comprising:

implanting or ingesting the device of feature 29 in the subject;

transmitting an ultrasonic signal in the subject at a focal point of the device; and

receiving an ultrasonic signal at an array of piezoelectric micromachined ultrasonic transducers operative to transduce a voltage from the ultrasonic signal, said voltage operative to indicate a condition in the subject.

40. The method of feature 38, wherein the ultrasonic signal comprises an ultrasound image.
41. The method of feature 39, wherein each of the piezoelectric micromachined ultrasonic transducers is operative to transduce a voltage from the ultrasonic signal at a pair of electrodes, each voltage operative to indicate a pixel of the ultrasound image.
42. The method of feature 39, wherein the condition is a rise in temperature of a biological tissue in the subject at a focal point of the device.

Alternatively, the technology can be summarized in the following alternative list of features.

A1. A system for ultrasonically heating biological tissue, comprising:

a device implantable in a subject's body, the device comprising:
a substrate, and
an array of piezoelectric micromachined ultrasonic transducers (pMUTs) supported on the substrate; and

a controller operative to control the array to emit and focus ultrasound transmissions at biological tissue in the body to heat the biological tissue.

A2. The system of feature A1, wherein each of said pMUTs comprises a layer of piezoelectric material sandwiched between two electrode layers, and wherein the substrate comprises an insulating layer between a base layer and one of the electrode layers.
A3. The system of feature A2, wherein each of the piezoelectric material layer and the insulating layer has a thickness from about 200 nm to about 5000 nm.
A4. The system of feature A2 or A3, wherein the base layer comprises silicon, the insulating layer comprises silicon dioxide, the electrode layers comprise gold or platinum, and the piezoelectric layer comprises aluminum nitride, scandium doped aluminum nitride, lithium niobate, or a combination thereof.
A5. The system of any of features A1-A4, wherein each pMUT of the array is independently addressable by the controller, and wherein the controller is operative to determine a focal point of ultrasound transmissions from the array by providing a time delay to an AC voltage applied to each pMUT of the array.
A6. The system of any of features A1-A5 comprising two or more of said arrays, wherein each array is in communication with the controller, which is operative to determine a common focal point of ultrasound transmissions from the two or more arrays.
A7. The system of any of features A1-A6, wherein the controller comprises a plurality of directly modulated ultrasound transducer circuits, each circuit comprising an inductor, a capacitor, a voltage input, and a bipolar junction transistor, and each circuit controlling operation of a different one of said array of pMUTs.
A8. The system of feature A7, further comprising a microprocessor in communication with the plurality of transducer circuits and operative to provide an input voltage to each of the transducer circuits.
A9. The system of any of features A1-A8, wherein the controller is implantable, or wherein said implantable device comprises the controller.
A10. The system of any of features A1-A9, wherein the pMUTs produce ultrasound transmissions at a frequency in the range from about 20 kHz to about 200 MHz.
A11. The system of any of features A1-A10, wherein the array comprises from 1×1 pMUT to about 200×200 pMUTs.
A12. The system of any of features A1-A11, wherein the system is capable, when implanted in a subject's body, of heating biological tissue of the subject from about 37° C. to at least about 41° C.
A13. An implantable, wearable, or portable medical device comprising the system of any of features A1-A12.
A14. A method of ultrasonically heating a biological tissue in a subject, the method comprising:

(a) implanting into the subject's body (i) a device comprising an array of pMUTs supported on a substrate, wherein the pMUTs of the array are in communication with a controller operative to control the pMUTs of the array to emit and focus ultrasound transmissions, or (ii) the system of any of features A1-A12, or the medical device of feature A13; and

(b) causing one or more of the pMUTs of the array to emit an ultrasound transmission focused on the biological tissue, thereby heating the tissue.

A15. The method of feature A14, wherein two or more of said arrays are implanted, each array in communication with the controller, which is operative to determine a common focal point of ultrasound transmissions from the two or more arrays.
A16. The method of feature A14 or A15, wherein the biological tissue is heated to at least about 41° C.
A17. The method of feature A16, wherein the biological tissue is heated to at least about 60° C.
A18. The method of any of features A14-A17, wherein the heated biological tissue comprises cancer cells.
A19. The method of feature A18, wherein the cancer cells are selected from the group consisting of neck cancer cells, brain cancer cells, thyroid cancer cells, breast cancer cells, prostate cancer cells, kidney cancer cells, endometrial cancer cells, pancreatic cancer cells, lung cancer cells, esophageal cancer cells, bladder cancer cells, rectal cancer cells, cervical cancer cells, ovarian cancer cells, peritoneal cancer cells, sarcoma cancer cells, neuroblastoma cancer cells, leukemia cancer cells, melanoma cancer cells, and combinations thereof.
A20. The method of any of features A14-A19, wherein the method is repeated one or more times, optionally with alteration of a focal point of the ultrasound transmissions.
A21. The method of any of features A14-A20, wherein the method is combined with one or more of radiation therapy, immunotherapy, targeted drug therapy, chemotherapy, radiofrequency therapy, imaging, or hormone therapy.
A22. The method of any of features A14-A21, wherein the method results in the death of cells of the biological tissue.

As used herein, the term “about” refers to a range of within plus or minus 10%, 5%, 1%, or 0.5% of the stated value.

As used herein, “consisting essentially of” allows the inclusion of materials or steps that do not materially affect the basic and novel characteristics of the claim. Any recitation herein of the term “comprising,” particularly in a description of components of a composition or in a description of elements of a device, can be exchanged with “consisting essentially of” or “consisting of.”

The present technology has been described in conjunction with certain preferred embodiments and aspects. It is to be understood that the technology is not limited to the exact details of construction, operation, exact materials or embodiments or aspects shown and described, and that various modifications, substitution of equivalents, alterations to the compositions, and other changes to the embodiments and aspects disclosed herein will be apparent to one of skill in the art.

### BRIEF DESCRIPTION OF THE DRAWINGS

FIG. 1 shows examples of implantable bio-heating systems (brain implant, thyroid implant, breast implant, kidney implant) based on pMUTs, compared to bulky extra-body high intensity focused ultrasound (HIFU) transducer devices (center). An illustration of a phased-array focusing technique for pMUT arrays is depicted at right.

FIG. 2 shows a finite element analysis (FEA) simulation in COMSOL Multiphysics® of the ultrasonic response of a pMUT array and the relative temperature response of a probe placed at the focal distance. The graph shows the temperature increase and decrease (scaled to the number of pMUTs used in the experiment) at different operation frequencies.

FIG. 3 shows an experimental setup for ultrasonic response measurement and ultrasonic heating response in a deionized water tank mimicking human tissue properties.

FIG. 4 shows ultrasonic response of a pMUT array at 700 kHz (top) and at 2 MHz (bottom). The array is driven with a Vₚₚ=2V burst signal of N=10 cycles sine waves. The received tone is measured at a distance of D=5 cm with a Teledyne hydrophone. The ultrasonic signal has an amplitude of about 54 mVₚₚ at 700 kHz and an amplitude of about 104 mVₚₚ at 2 MHz.

FIG. 5 shows experimental results of ultrasonic heating (measured with a thermocouple probe) at different frequencies: 700 kHz, 1 MHz and 2 MHz (Device A, thermocouple probe aligned with pMUT chip, Example 2).

FIG. 6 shows experimental results of ultrasonic heating (measured with a thermocouple probe misaligned with the pMUT chip) at different frequencies: 700 kHz, 1 MHz and 2 MHz (Device B, thermocouple probe misaligned with pMUT chip, Example 2).

FIG. 7 shows an example diagram of composition of a pMUT array.

FIG. 8 shows example steps of fabrication of a pMUT array.

FIG. 9 shows an example of a working principle of a single pMUT.

FIG. 10 shows an example of a directly modulated ultrasonic transducer (DMUT) electrical circuit diagram.

FIG. 11A shows an example of an array of the DMUT circuits (from FIG. 10) in an electrical circuit diagram. FIG. 11B shows an example photo of a printed circuit board (PCB) of a DMUT array on a PCB connected to a Teensy 3.6 microcontroller board that can pilot it.

FIG. 11C shows an example photo of a fabricated 10×5 pMUT array with rows 1-5 labeled and columns 0, 4, and 9 highlighted.

FIG. 12A shows plots of phased-array delays implemented with a Teensy 3.6 microcontroller board; described in Example 3. FIG. 12B shows plots of DMUT/pMUT array outputs relative to the delayed input signals generated (shown in FIG. 12A) with the micro-controller board.

FIG. 13 shows a photo of a 10×5 fabricated pMUT array and a Teledyne hydrophone submerged in a silicone oil tank and fixed at 5 mm for experimental measurements.

FIG. 14A shows a mathematical model of the Sound Pressure Level (SPL) for an unphased pMUT array at 5 mm FIG. 14B shows a mathematical model of the SPL for a phased and focused pMUT array at 5 mm FIG. 14C shows a measured received acoustic signal on a hydrophone with a pMUT phased array (time delayed input signals) off. FIG. 14D shows a measured received acoustic signal on a hydrophone demonstrating a 13 dB measured improvement of the SPL when using a DMUT/pMUT phased array (time delayed input signals) on.

FIG. 15A shows an example of an External Acoustic Transducer (EAT) and an Acoustic Discovery Architecture (ADA) for real time monitoring of Intrabody Networks and monitoring of Implanted Medical Devices (IMDs). FIG. 15B shows a diagram of an ADA working principle with two IMDs labeled in communication with pMUT arrays.

FIG. 16A shows an example ADA scanning setup with four chips of pMUT arrays placed on a PCB at the vertices of a square of side d; each of the four chips can be driven with a phased array technique in order to focus the energy at different focal points in the scanning area for acquiring data. FIG. 16B shows a photo of an STMicroelectonics Steval-IME011V2 pulser board which can be used to drive the four chips (FIG. 16A) at their resonance frequency and to program time delays (for a phased array technique). FIG. 16C shows a silicone oil tank used as transmission medium to emulate living tissue properties. Both the transmitter (PCB board with pMUT arrays performing phased arrays technique) and a receiver (a commercial Teledyne TC4038 hydrophone at center of FIG. 16C mimicking an IMD) are shown submerged in the silicon oil tank.

FIG. 17A shows example steps of a scanning algorithm implemented by an ADA. FIG. 17B shows OMNET++ (a discrete time network simulator based on C++ programming) ADA main modules based on an ultrasonic communication link. The main modules are: External Sensor, which represents a phased array platform with pMUT chips, the Body Channel which emulates living tissue as a communication medium, and the Internal Sensor, which models multiple IMDs placed at randomized positions inside a body torso. The main exchanged messages during the ADA protocol are the information request beacon (IRB) and the acknowledgement (ACK) Info.

FIG. 18A shows a fabricated pMUT array to achieve higher levels of acoustic power wherein individual pMUTs are laid in rectangular lattice arrays and electrically connected in parallel; N=45 columns and M=50 rows are shown with a scale bar of 500 μm, and the enlarged image has a scale bar of 100 μm. FIG. 18B shows a diagram of a phased array working principle. When multiple acoustic sources are used (e.g., four pMUT arrays), the generated acoustic waves will be subject to interference. In order to achieve maximum efficiency and constructive interference at a certain point in space (a focal point F), all the acoustic waves need to be about in phase at F. The wave interference illustrates each source (pMUT array) is driven with a certain delay with respect to the closest source to the focal point F.

FIGS. 19A-19D show acoustic signal measurements on the receiver placed 5 cm from the transmitter for 4 of the pMUT chips shown in FIG. 18A. Measurements were taken both with and without the use of a beam steering, phased array technique.

FIGS. 20A-20D show an ADA's simulation results based on the phased array steering experimental results. FIG. 20A shows the discovery time for ten IMDs while sweeping the scanning accuracy (scanRange). FIG. 20B shows the discovery energy versus scanDelta while sweeping the accuracy. FIG. 20C shows percentage of discovered nodes versus the scanning range percentage (scanRange), which is the ratio focused beam area and the IMD size, for different scanDelta. FIG. 20D shows energy consumption versus the number of nodes/IMDs in the simulated IN.

FIG. 21 shows an illustration of an angle limit a of an ultrasonic beam.

FIGS. 22A-22B show an illustration of a scanning protocol with an external ultrasonic scanner or ultrasonic stethoscope. FIG. 22A illustrates a first scan central to the body torso to find the position of the IMDs. FIG. 22B illustrates once the IMDs have been found, an ultrasonic stethoscope can be moved on top of the device (i.e. IMD 2) of interest to communicate with; at the end of the scanning protocol, all the positions of the devices have been detected and the stethoscope can be moved on top of the IMD of interest to improve the ultrasonic communication link (both for data and power transfer).

FIG. 23 shows an optical image of a pMUT array example for acoustic communication links. The scale bar at lower left is 200 μm. The radius of one pMUT (r_{pMUT}) is 30 μm.

FIG. 24 shows a model of array directivity and sensitivity for the array shown in FIG. 23.

FIG. 25A shows an illustration of information encoding including pixel serialization into a bit stream. FIG. 25B shows an illustration of a QPSK (Quadrature Phase-Shift Keying) communication scheme designed and fed with serialized pixels from a transmitted image over a channel. FIG. 25C shows a testing setup of an ultrasonic QPSK transceiver.

FIG. 26A shows data (image) transmission at 3.5 cm. FIG. 26B shows data (image) transmission at 13.5 cm.

### DETAILED DESCRIPTION

The technology described herein provides miniaturized ultrasonic transducers that can be implanted in a body (human, animal, unknown species, etc.) and help treat tissue, cells, organs or other parts. The ultrasonic transducers are able to generate sound waves, beyond hearing range, commonly denoted as ultrasounds. The generated ultrasounds can be absorbed and the energy converted into heat. Depending on the intensity of the waves, this can just heat up some regions of the body or even ablate targeted regions. The technology can be used for continuous treatment and monitoring of a patient's body in its general definition, as a form of ultrasound therapy, and overcomes the need for bulky focused ultrasound transducers, from outside the body.

The technology overcomes disadvantages of prior technologies that are invasive for patients and cannot be used for continuous treatment or monitoring, or that cannot be used outside hospital and clinic facilities. The present technology has the advantage of being miniaturizable and biocompatible, therefore implantable into a body. Furthermore, in the technology, the ultrasonic transducers can be fabricated in arrays which gives them the flexibility to re-configure electronically the focus of the ultrasonic energy without moving the device. This can overcome disadvantages of fabrication with a fixed focus point, in which the device needs to be mechanically moved to focus in different points. The present technology, when implanted in a body, can be more precise and more effective than technologies that act from outside the body.

Ultrasound beams produced by extra-body transducers can be used for therapeutic applications. Typical extra-body transducers are applied externally to a subject while the subject remains still. When the ultrasonic beams reach a tissue volume, part of the energy is absorbed and converted into heat. The increase in temperature depends on the physical properties of the medium, such as its absorption coefficient, density and specific heat, the ultrasound properties, such as frequency and intensity, and ultimately on the geometry of the tissue. Two application categories include ultrasound hyperthermia and focused ultrasound surgery or ultrasound ablation. The first category is a long but reversible therapy that can last from thirty to sixty minutes, increasing the temperature up to about 41° C. to 45° C. The second category is a short (about thirty seconds) but high intensity focused ultrasound procedure that can bring the tissue up to greater than about 50-60° C. or up to about 90° C., creating permanent biological change. This thermal ablation rapidly heats cancerous tissue to temperatures greater than about 50-60° C., which are sufficient temperatures for example, for coagulative necrosis. The implantable technology herein can provide rapid heating and can be utilized for both categories.

The technology provides implantable bio-heating systems based on piezoelectric micromachined ultrasonic transducers (pMUTs). The technology can provide a device including a pMUT array of radiating elements that can focus ultrasonic energy to heat up tissue. The pMUTs can use aluminum nitride (AlN) as a piezoelectric material for actuation, which is a biocompatible material. In some examples, the AlN can be further optimized by doping it with scandium (ScAlN). Other piezoelectric materials can be used. The pMUTs can be designed in arrays that allow electronic re-configuration of a focal point for the array and targeting of different regions to heat. The device can be implanted and act as a bio-heating system, for example, for hyperthermia therapies.

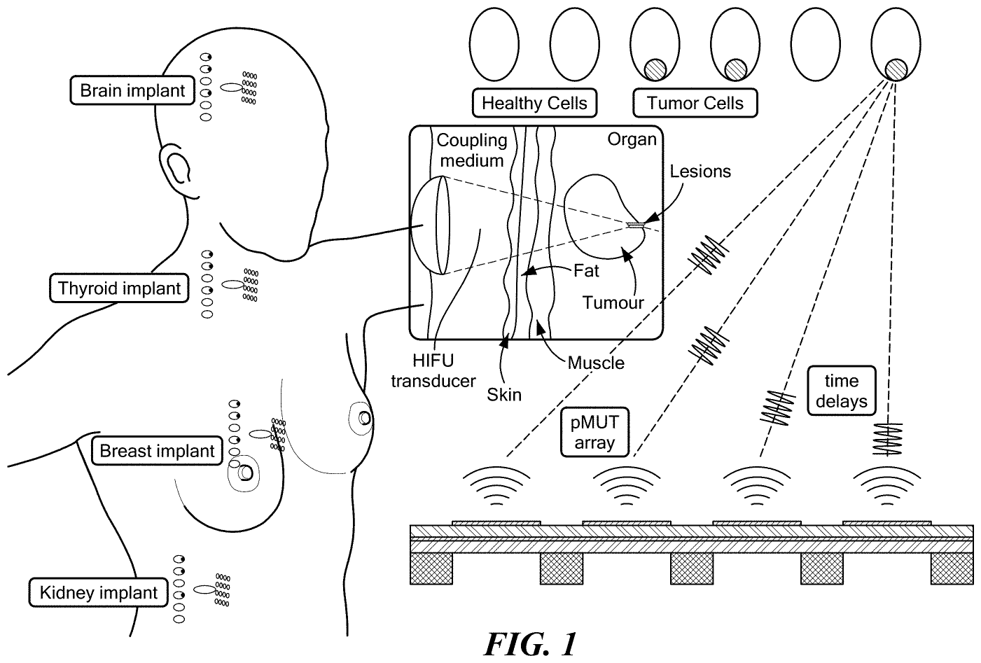

The technology can be implemented as an ultrasonic heating system based on pMUTS in a variety of ways. The pMUT arrays are biocompatible and can be miniaturized and implanted, allowing a versatile system to focus the ultrasonic energy in a target area and heat the tissue. FIG. 1 shows an implantable bio-heating system based on pMUTs. At the left of FIG. 1, examples of a brain implant, a thyroid implant, a breast implant, and a kidney implant are depicted. Compared to bulky extra-body High Intensity Focused Ultrasound (HIFU transducer, FIG. 1, center) devices, the pMUTs can enable non-invasive continuous treatment and monitoring of patients by using intrabody communication links. An illustration of a phased-array or beam focusing technique applied to tumor cells is shown at the right of FIG. 1.

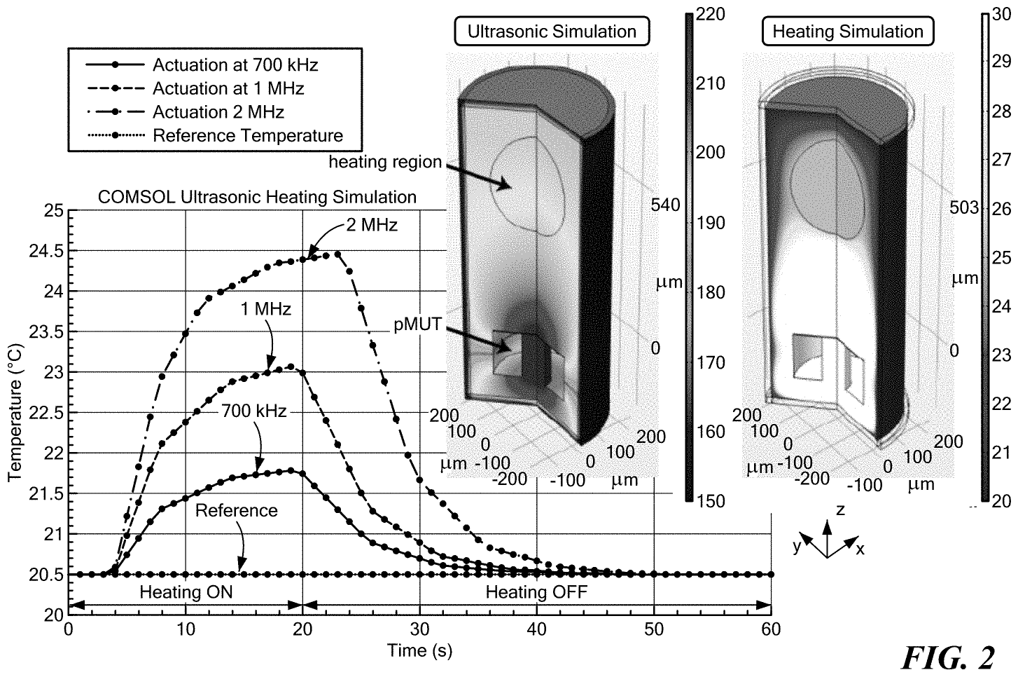

To simulate a pMUT scenario for ultrasonic heating, COMSOL Multiphysics® was chosen, given its capability of integrating multiple physical domains. The results of an ultrasonic and heating simulation are shown in FIG. 2. The simulated pMUT is driven at three different frequencies (700 KHz, 1 MHz, and 2 MHz), and the heating curves are shown in the plot. In the first period the pMUT is actuated; thus the heating is ON, and an increase in temperature is observed, starting from ambient temperature of 20.5° C. and increasing to 21.5° C., 23° C. and 24.5° C. respectively. At this point, once the pMUT array is deactivated, heating is OFF, and the temperature decreases exponentially.

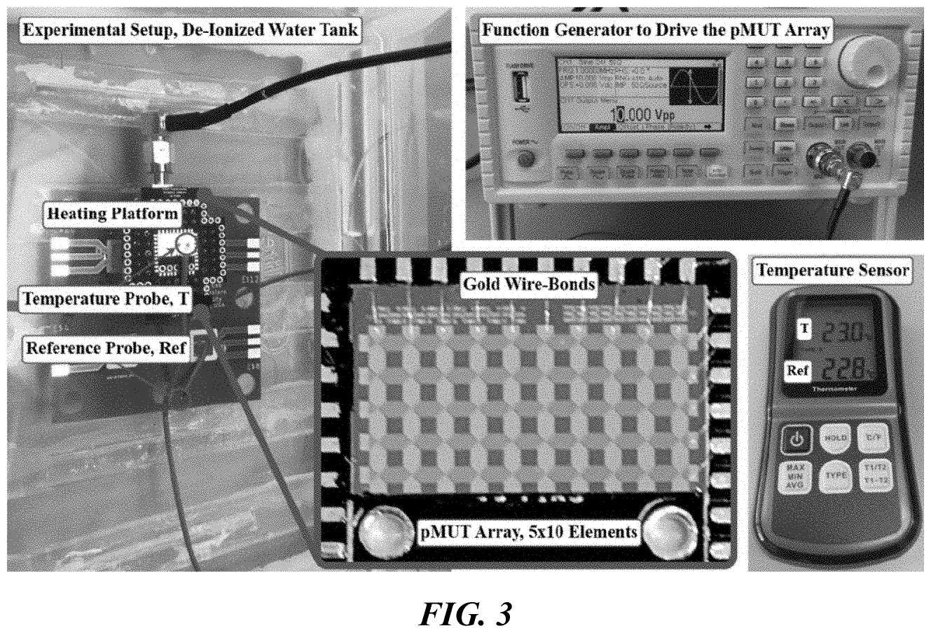

To implement an array of pMUTs for heating, for example, as shown at the center of FIG. 3, a five by ten elements pMUT array can be wire-bonded to a circuit board and submerged in a deionized water tank (left, FIG. 3), mimicking human tissue properties and isolating possible electrical conduction paths. An example of a function generator to drive the pMUT array is shown at the top right of FIG. 3, and an example of a temperature sensor is shown at the bottom right of FIG. 3.

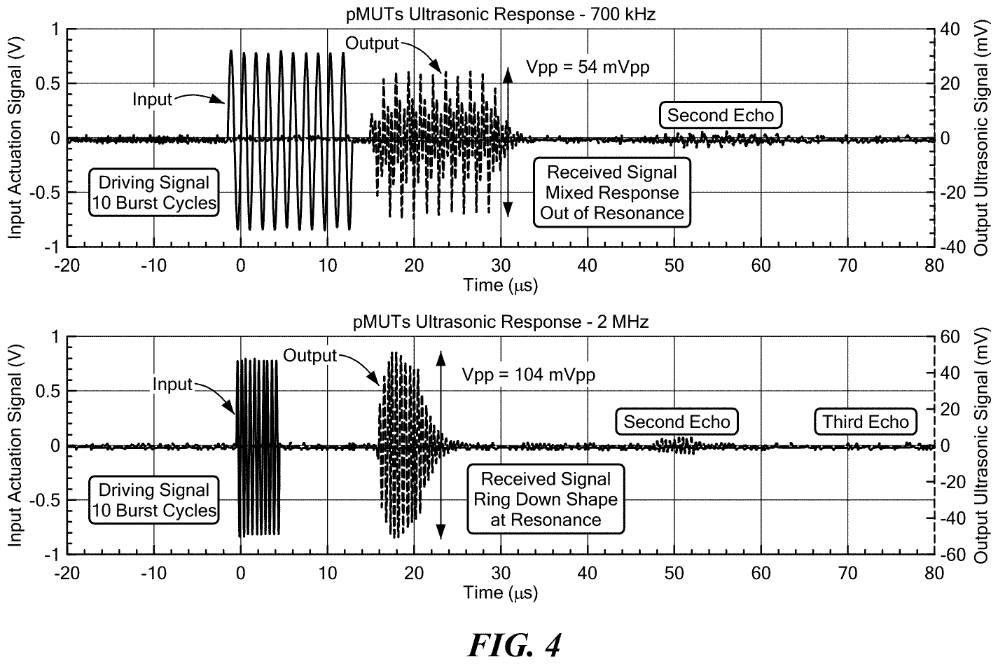

For example, the pMUTs in a 5×10 array (bottom, FIG. 3) can be driven with a 2V peak-to-peak burst signal of ten sine wave cycles as shown in the inputs labeled in the plots of FIG. 4. Referring to FIG. 4, the top plot shows pMUT ultrasonic response at about 700 kHz, and the bottom plot shows pMUT ultrasonic response at about 2 MHz. The ultrasonic response is measured with a commercial Teledyne hydrophone. The measured outputs are shown in the outputs of FIG. 4.

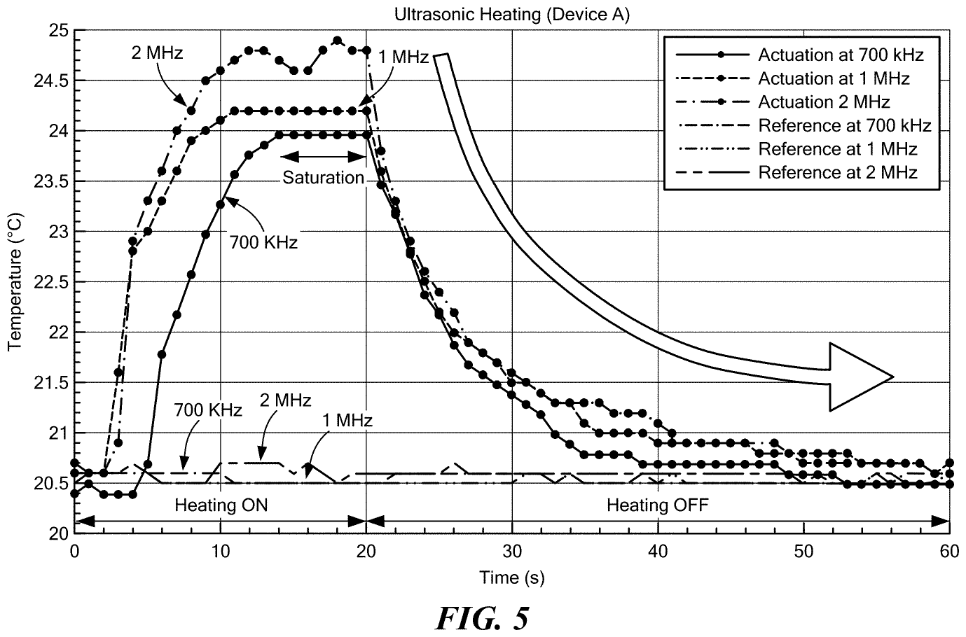

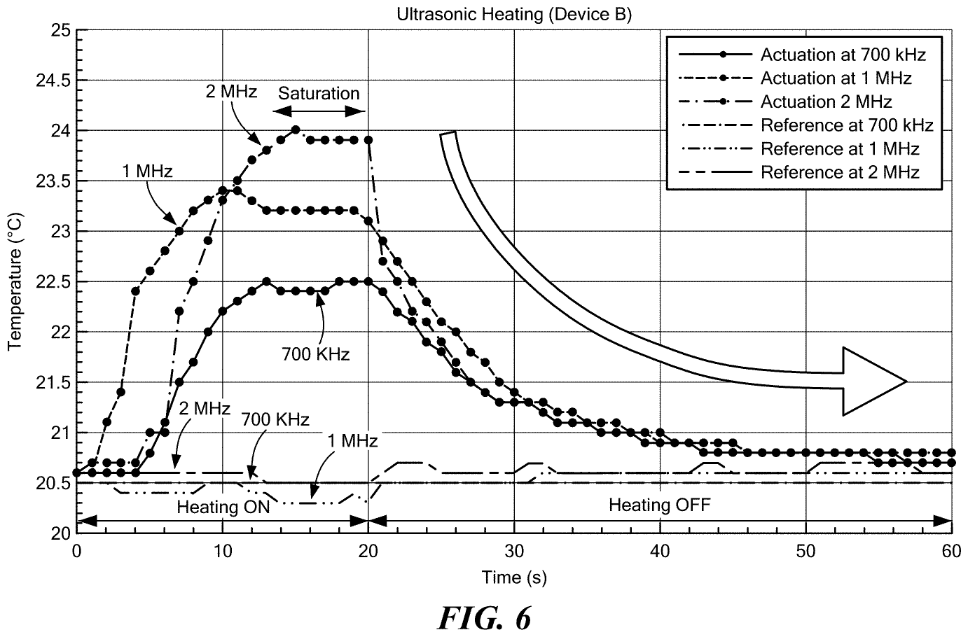

The heating process can be measured with thermocouple probes. The pMUT array of 5×10 elements is used to heat the thermocouple probes in a tissue-like environment. In the plots presented in FIG. 5 and in FIG. 6, both the array and the probe are submerged in a DI water tank to emulate the ultrasonic properties of the human body. The devices are driven with a burst signal of ten sine waves at three different frequencies. The heating process is monitored over time. The two different devices are tested for their heating capabilities at five centimeters. The probe tip is placed at the focal distance of the array in order to maximize the acoustic energy. One thermocouple probe, Device A of FIG. 5, is aligned with the pMUT array to monitor the heating of the probe, while a second thermocouple probe, Device B of FIG. 6, is misaligned with the pMUT array. The individual pMUT elements are actuated in parallel during these measurements. The heating results of the probes are collected over time at multiple frequencies, showing good matching with the simulation results from FIG. 2. This Device B pMUT array (FIG. 6) shows slightly lower performance compared to Device A due to the misalignment of the measuring probe. Once the driving signal is switched on, there is a latency for the heating of about 2-4 seconds. After about 10 seconds, the temperature saturates. At 20 seconds the ultrasonic signal is turned off and the temperature decays according to the equivalent thermal loss coefficient of the probe and propagation medium (DI water). The experiment shows temperature increments of up to 4° C. relative to the medium temperature. When the device is implanted into a human body, this can provide local heating of tissues from 37° C. to 41° C., making it useful for hyperthermia therapies.

Given the biocompatibility and miniaturization capability of the pMUT arrays, this system can be employed as a continuous micro-therapy system. For example, the pMUT arrays can be implanted and monitored. The technology can provide miniaturized implantable arrays of piezoelectric micro-machined ultrasonic transducers (pMUTs). Each device can be a micro-fabricated membrane on top of a cavity. One example stack of materials for the membrane can be the following: a supporting layer such as silicon dioxide (SiO₂) or native Silicon (Si), and a piezoelectric layer, such as aluminum nitride (AlN), scandium doped AlN (ScAlN), lithium niobate (LN), for example, sandwiched between a top and bottom electrode, which can be platinum (Pt), aluminum (A1), gold (Au), or other suitable conductive material. The piezoelectric layer can be also activated from one layer of metal as well (both electrodes on the same metal).

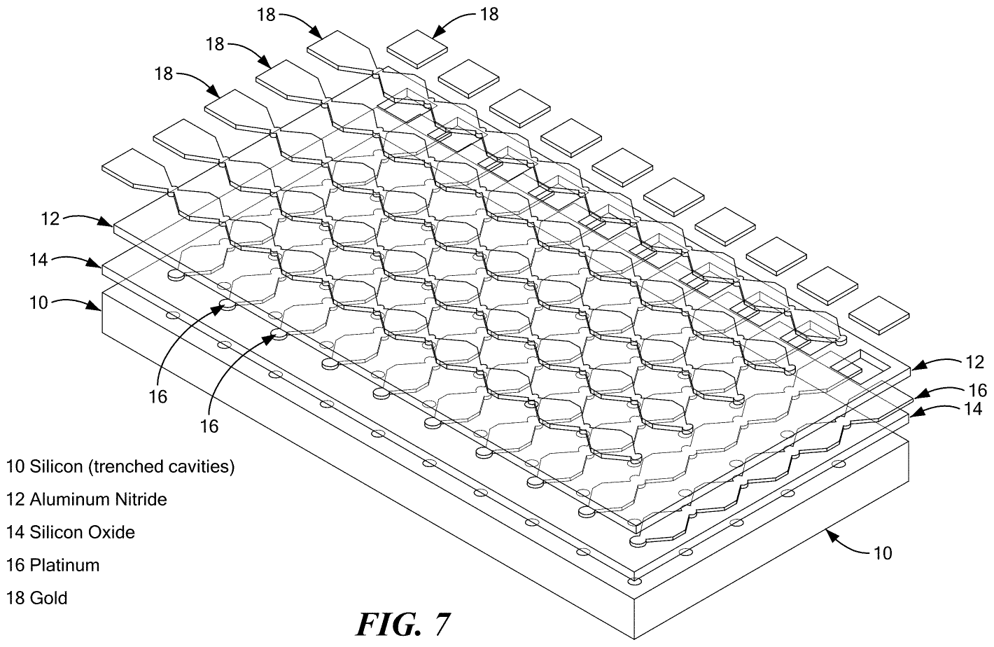

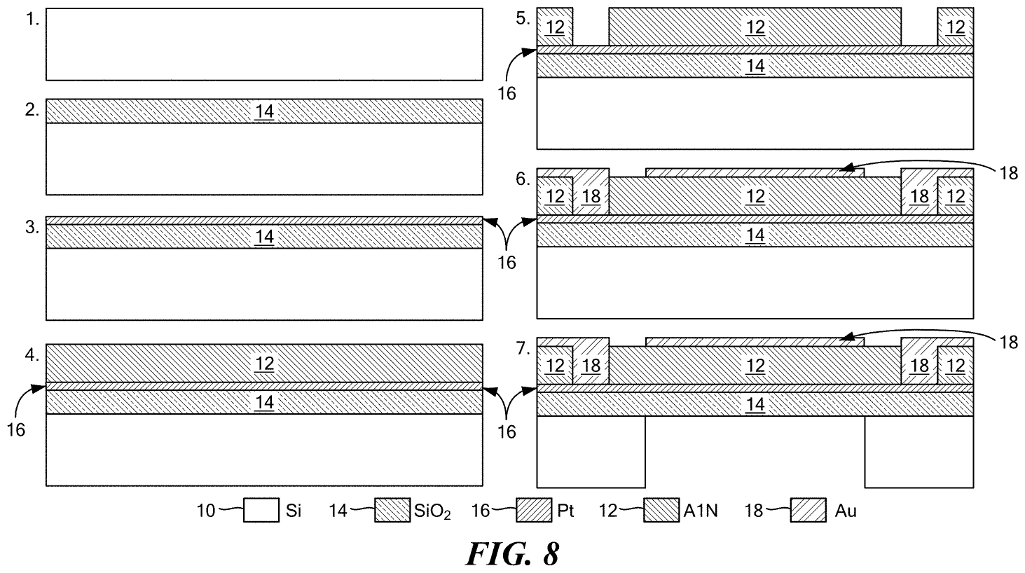

Referring to FIG. 7, an example breakdown of a composition of a pMUT array is shown. The top electrode is 200 nm of gold (Au). The piezoelectric layer is 1 μm of aluminum nitride (AlN) and it has been etched to access the bottom layer (vias). The bottom electrode is 200 nm of platinum (Pt) and it sits on a structural layer made of 1 μm of silicon oxide (SiO). All these layers sit on top of a 300 μm silicon substrate where the pMUT cavities have been trenched with deep reactive ion etching (DRIE) technique. Referring to FIG. 8, an example fabrication process can start with a double side polished silicon wafer of 300 μm. Following, a layer of silicon dioxide of 1 μm can be deposited. Then 200 nm of platinum can be sputtered as a bottom electrode and patterned through a lift-off process. The piezoelectric material, in this example aluminum nitride, can then be deposited to reach 1 μm. At this point, in order to access to the bottom electrode, the nitride (AlN) layer can be etched with hot phosphoric acid. On top of the nitride, a 200 nm layer of gold can be sputtered as a top electrode and patterned through a lift-off process. A hard mask layer can be used to pattern the cavities of the pMUTs on the back of the wafer. The devices can be released with deep reactive-ion etching (DRIE).

For example, the pMUT arrays can be micro-fabricated in 8-inch industrial foundries. Each wafer can contain hundreds or thousands of pMUT devices. A single bare chip can be made cost effectively.

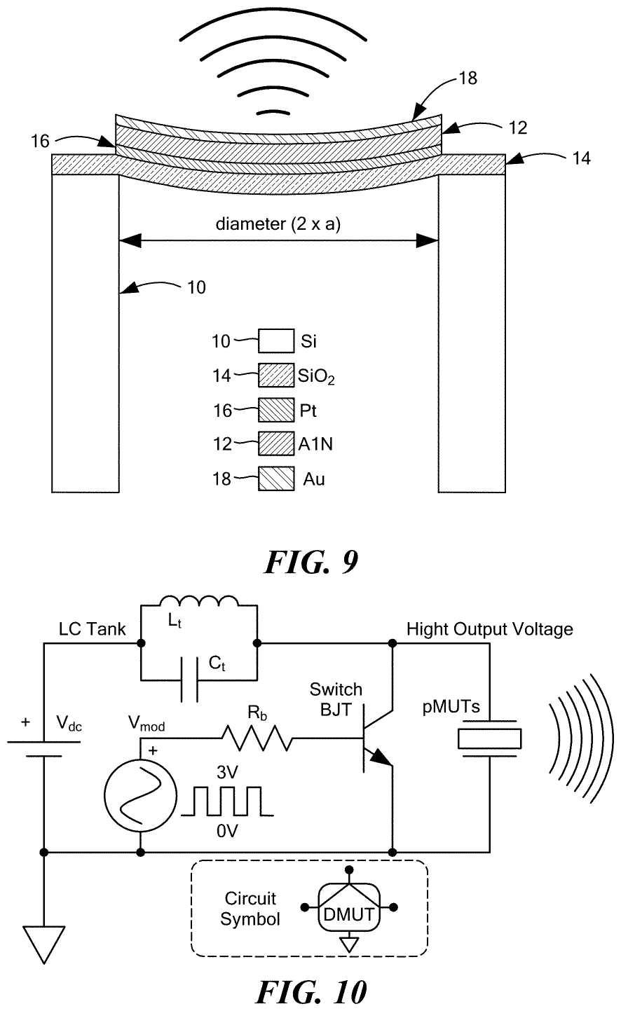

When applying a voltage between two different electrodes (for example top and bottom), and due to the piezoelectric effect, the membrane can start pushing against the walls of the cavity. Given this boundary condition (the cavity), the membrane can start vibrating in the perpendicular direction (Z-axis). This vibration of the membrane can generate ultrasonic waves that can propagate into the medium (e.g., air, water, de-ionized water, living tissue, tissue phantom). Depending on the configuration of the pMUT, frequency response can be in ranges useful for hyperthermia or for imaging. FIG. 9 shows an example illustration of a working principle of a single pMUT. For example, one single membrane can generate sound waves mostly in every direction, making it an omnidirectional radiating element.

When designing multiple membranes/pMUTs in an array, the ultrasonic waves generated by each element can start combining together. Depending on the relative phase-shift of all the combined waves, at each point in space, those can add up (constructive interference) or subtract (destructive interference). Each array, depending mostly on the pitch (the relative distance between individual elements), has a natural focal point, where most of the ultrasonic waves interact in a constructive way. As is describe herein, it is possible to drive each element of the array with different delays and to change this combined focal point in space, using a phased-array and beam-forming technique. Multiple arrays can each be focused to provide advantages, for example, less complex electronics, higher SPL, focused heating, scanning, and broader coverage. The devices can be controlled with any suitable electronics. Processing tasks can be carried out by one or more processors and memory, for example to implement driving of each element of the array as described herein. The pMUT control circuitry can be designed to be easily compatible with a typical voltage output (e.g., about <3.5 V) from a microprocessor.

For example, a Directly Modulated Ultrasonic Transducer (DMUT) electrical circuit (FIG. 10) can be utilized in a phased-array platform. The DMUT circuit allows to directly feed an ON/OFF keying signal into the transducer and at the same time boost the voltage on top of it, improving the output pressure and signal to noise ratio (SNR). The DMUT circuit can be utilized for its voltage boosting capability and to take advantage of the low input signal. For example, easily obtained transistor-transistor logic boards/chips nearing output stage of about <3.5V can lower the cost of electronics utilized. The example circuit (FIG. 10) can be driven with input signals of less than about 3.5V, which allows for its integration with commercial micro-controllers. This allows piloting of multiple DMUT circuits and control of their phase-shifting (or time-delay) in order to implement the phased-array technique. The circuit shown in FIG. 10 includes a Bipolar Junction Transistor (BJT) acting as a switch when driven in its cut-off region (low voltage) and saturation region (high voltage). By applying a train of pulses at the base of the transistor, this will connect and disconnect a DC biased LC tank to the acoustic transducer. When modulating the switch with an ON/OFF signal, the LC tank will abruptly change the resonance frequency due to the high capacitance of the pMUT array. In this way, a portion of the energy stored by the inductance of the LC filter will be stored by the transducer. This mechanism can explain the generation of the high voltages at the output of the DMUT system. The inductance and capacitance of the LC tank can be changed to provide different output frequencies.

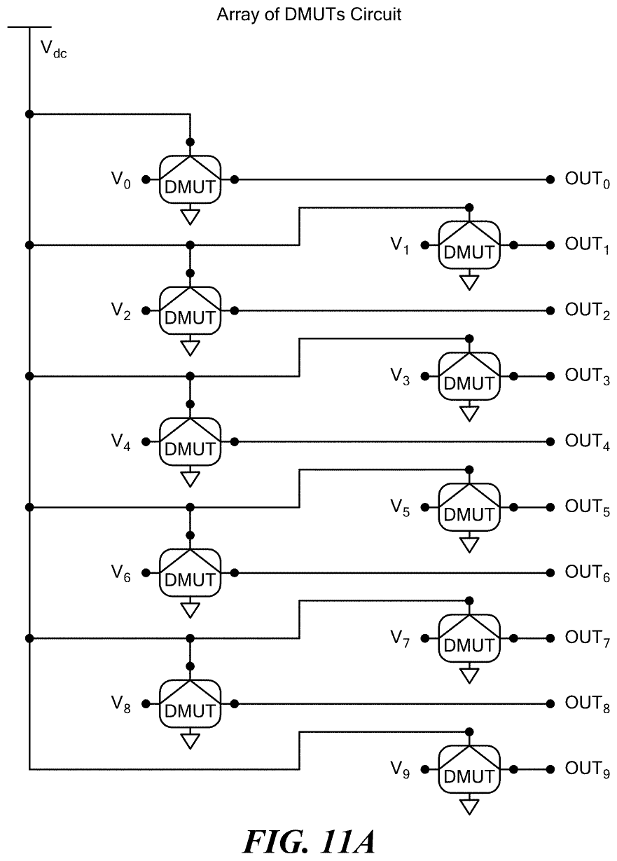

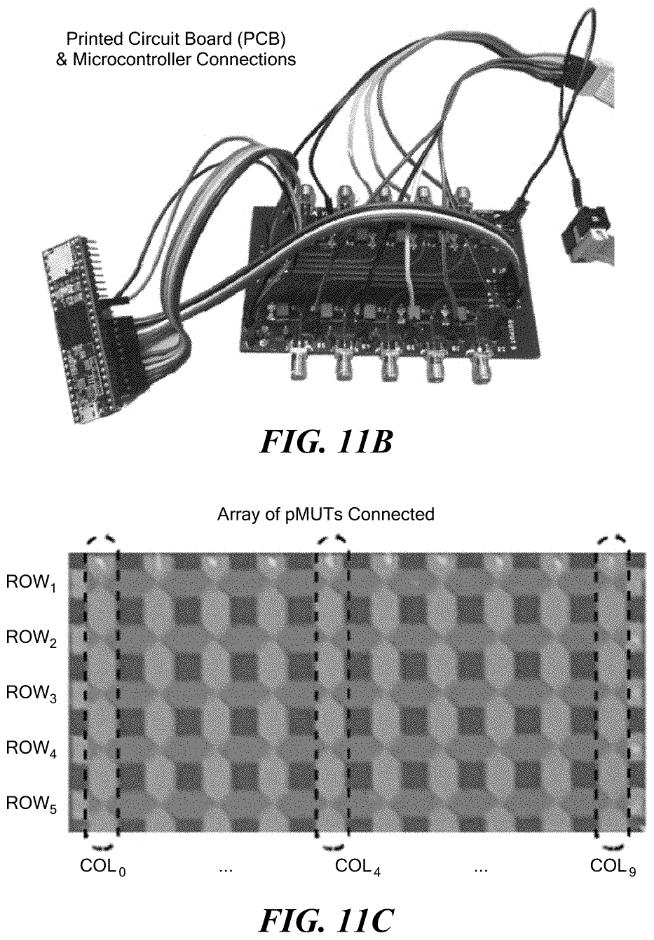

As illustrated in FIG. 11A, the single DMUT circuit shown in FIG. 10 can be laid out in arrays (e.g., on a PCB) in order to pilot individual channels of a pMUT array. The example shown in FIG. 11A has output channels 0-9. Hundreds or thousands of output channels can be configured on a PCB or on an implantable support. An example of a fabricated PCB and the connections to a Teensy 3.6 micro-controller to pilot an array are shown in FIG. 11B. The Teensy 3.6 microcontroller can be easily acquired and configured. An optical image of a fabricated pMUT array is shown in FIG. 11C. FIG. 11C highlights the rows one to five (connected to ground) and the columns zero-nine (connected to the outputs zero-nine of the DMUT array illustrated in FIG. 11A).

When placing an object, tissue, organ, cell, etc., in front of a pMUT array, part of the ultrasonic energy can be absorbed and transformed into heat. If placing those objects, tissues, organs, cells, etc., at the focal point (either the natural focal point or the phased-array/beam-formed one), this heating effect can be maximized. The focal point of the pMUT array (or of more than one pMUT array) can be directed, for example, to a small treatment area, while minimizing consequential damage to surrounding healthy cells. Depending on the sound pressure level (SPL) at the focal point, the shape and thermal absorption coefficient of the object, tissue, organ, cell, etc., and the medium properties (such as density, speed of sound, attenuation, and absorption coefficient), increases in the temperature of the object can result.

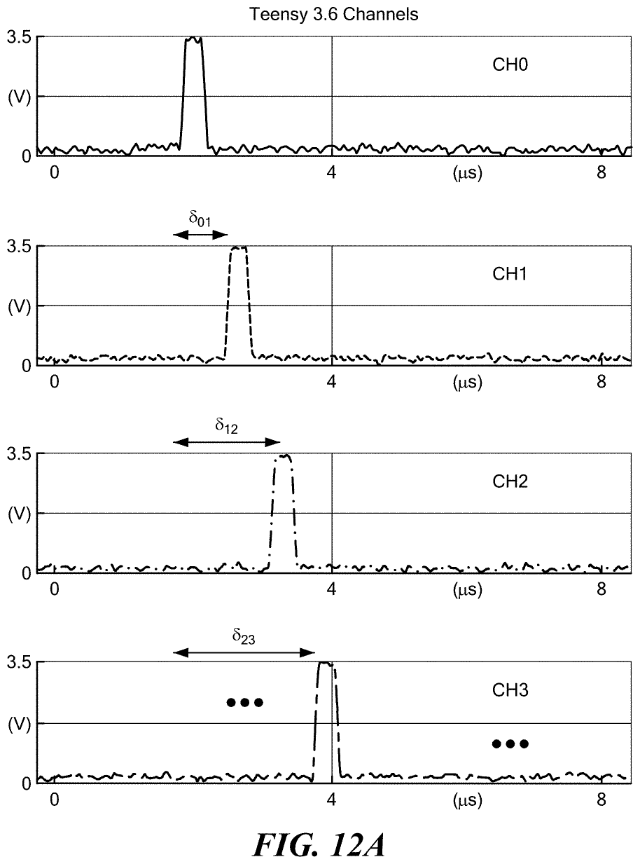

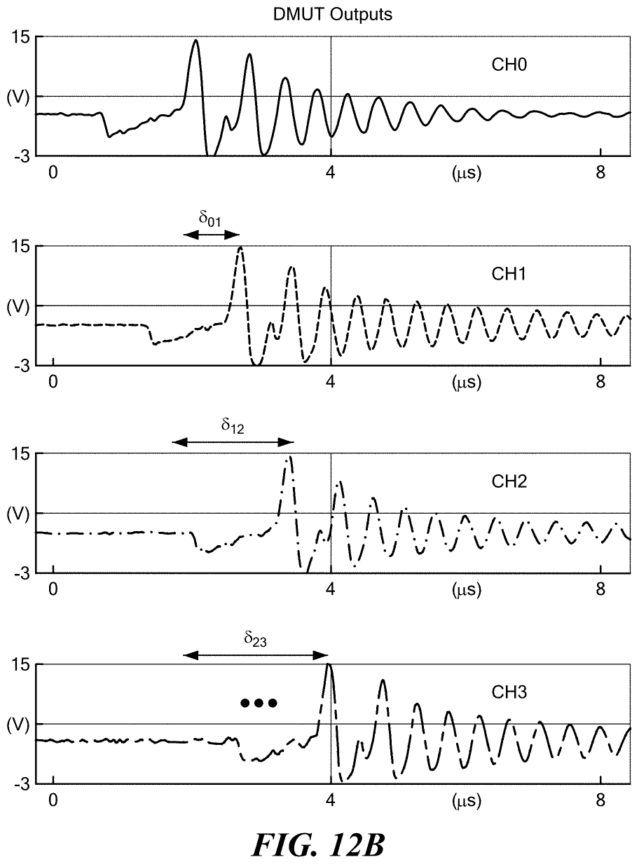

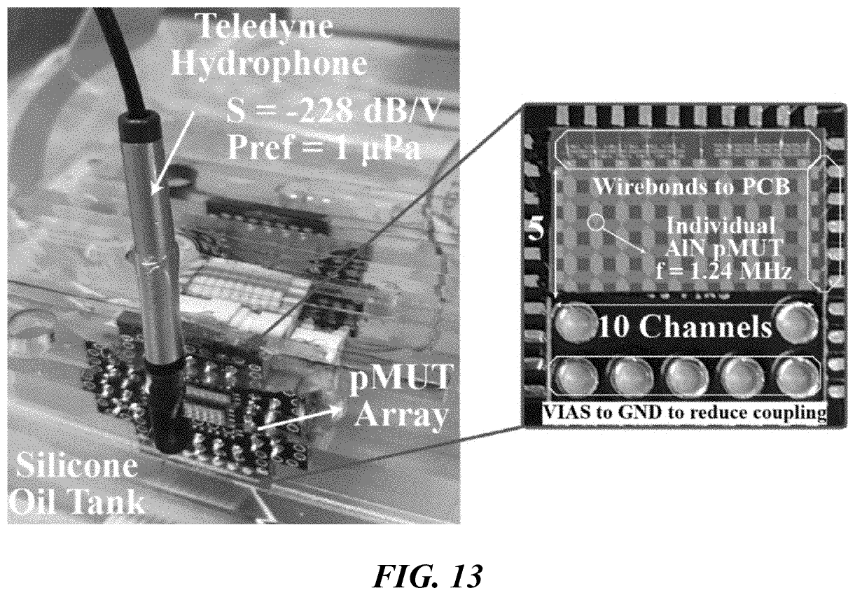

An objective of the phased-array or beam focusing technique is to generate constructive interference of the ultrasonic waves at a certain focal point in space. This allows to have a higher acoustic signal, improve the transmission distance and the SNR. Each individual pMUT in an array can be focused at a focal point by delaying or timing the signals in order for the ultrasound to arrive at a desired phase from each pMUT. The focusing can be achieved by delaying the signals of different columns of the pMUT array in order for the ultrasonic signal to arrive in phase at the desired distance from the array. By doing so, the waves add up constructively instead of creating destructive interference. Example driving signals implemented in the micro-controller (e.g., the microcontroller shown at left of FIG. 11B) are shown in FIG. 12A. In FIG. 12A, time in microseconds is shown on the X-axis for channels 0-3. The relative outputs of the DMUT array, which connect directly to the columns of the pMUT array, are shown in FIG. 12B. As shown in FIG. 12B, constructive interference waveforms can be generated. An example configuration to test the output of entire phased array with a hydrophone is depicted at the left of FIG. 13.

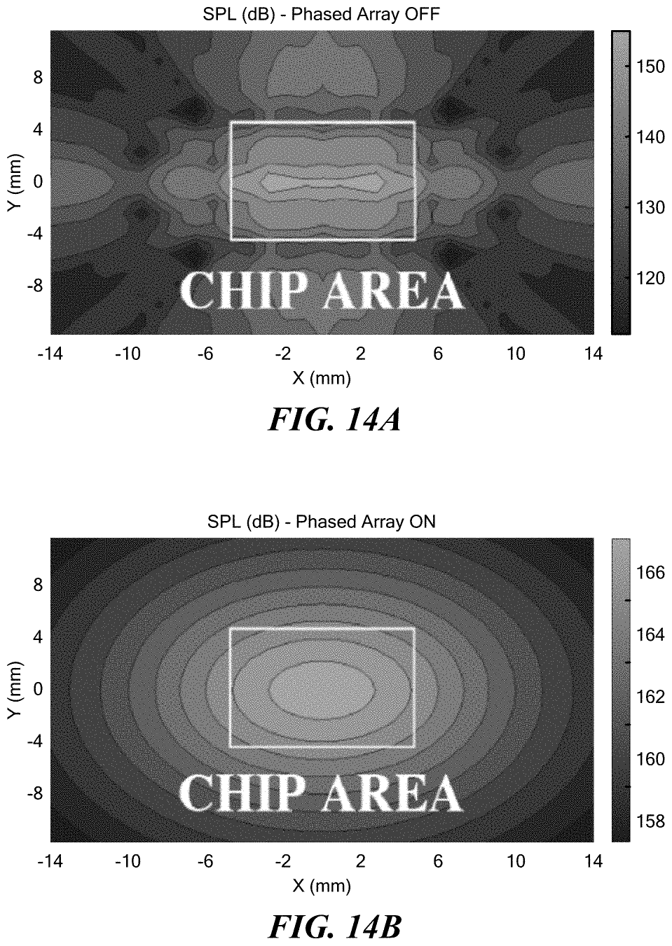

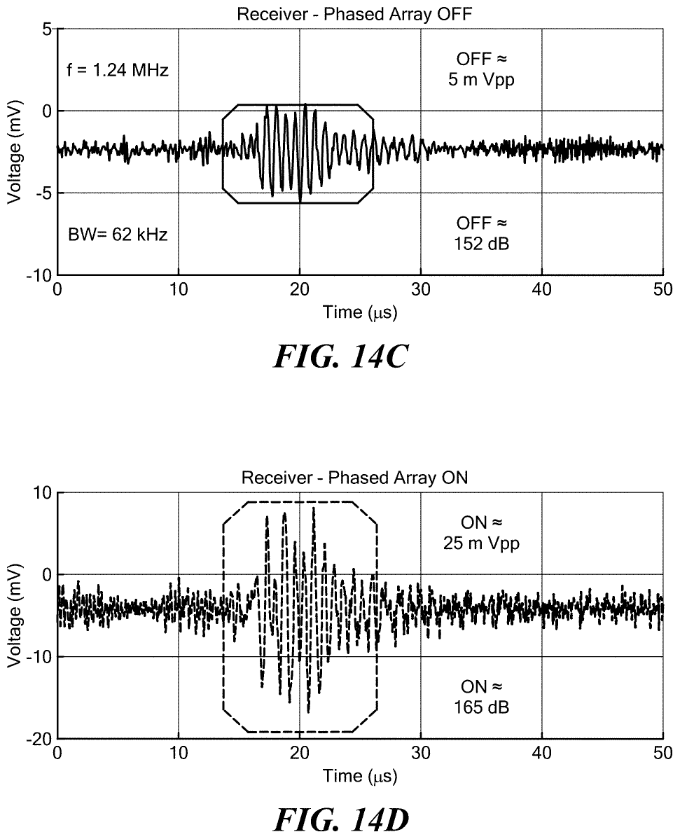

Modeling of the effectiveness of the phased-array technique is studied, starting with constructs from a Digital Holographic Microscope, and is presented in the Examples. For example, a mathematical model of the SPL for a pMUT array, shown at the right of FIG. 13, is presented in FIG. 14A. In FIG. 14A, the phased array technique described above is off. When the phased array technique is off, destructive interference occurs. In FIG. 14A, the SPL legend is shown at right, with lighter shades indicating higher SPLs up to about 150 dB. In FIG. 14B, the phased array, including the timing of the driving signals is on, and a higher focused SPL is achieved with the phased array on. The lighter shades indicate a focused SPL up to about 166 dB.

Examples of empirical results are presented in FIG. 14C and in FIG. 14D. In FIG. 14C, with the phased array off, 5 mVpp (about 152 dB) is achieved. In FIG. 14D, with the phased array on, 25 mVpp (about 165 dB) is achieved. The data demonstrates that focused temperatures higher than about 41° C., higher than about 45° C., higher than about 50° C., or higher than about 60° C. can be achieved. The focused temperatures can be achieved rapidly (e.g., FIG. 5). For example, the focused temperatures can be achieved in less than about 1 second, less than about 2 seconds, less than about 5 seconds, or less than about 10 seconds.

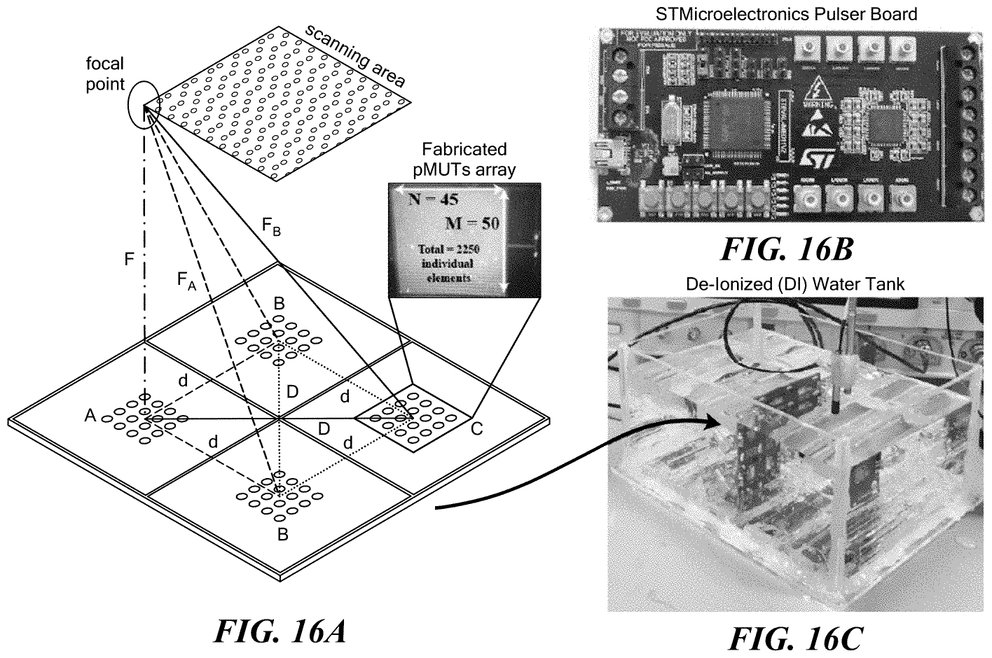

Examples described below can demonstrate the combining of two or more arrays of pMUTs in a phased array or beam focusing technique to achieve, for example, higher focused SPL. When applied to hyperthermia, implementation of a phased array can increase focus of SPL level, for example, by using the example circuit presented in FIG. 11A connected to an array of pMUTs (FIGS. 11B, 11C). The arrays of pMUTs disclosed herein can be larger or smaller arrays. Arrays of pMUTs can be strategically combined. For example, each array of pMUTs can be configured with distances (e.g., “D” and “d” depicted in FIG. 16A) between arrays.

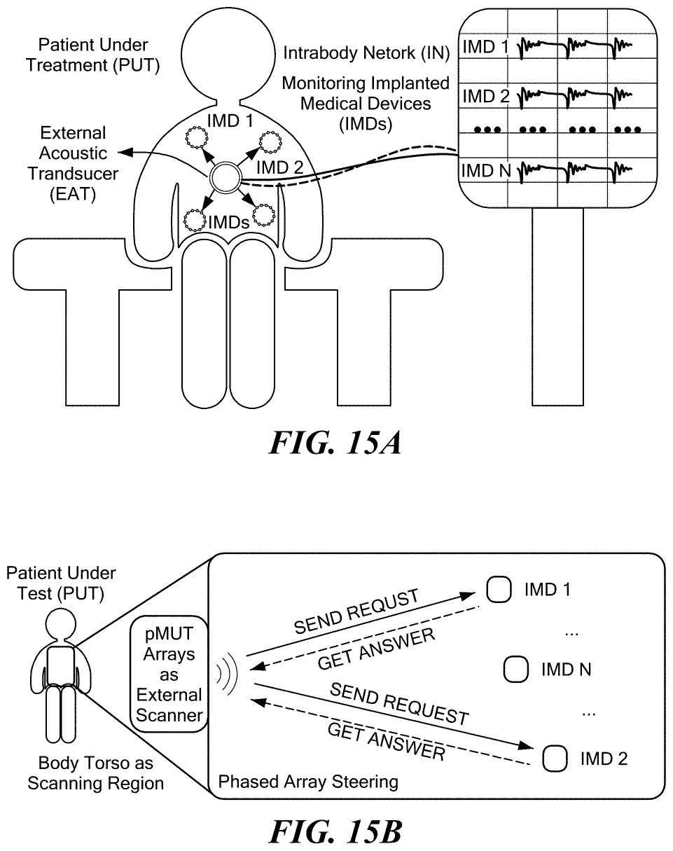

The pMUT arrays can be utilized in internal or external discovery architecture. As shown in FIG. 15A, an example of an External Acoustic Transducer (EAT) uses an Acoustic Discovery Architecture (ADA) for real time monitoring of Intrabody Networks (INs) and monitoring of Implanted Medical Devices (IMDs). FIG. 15B shows an example of an ADA working principle with two IMDs labeled in communication with pMUT arrays.

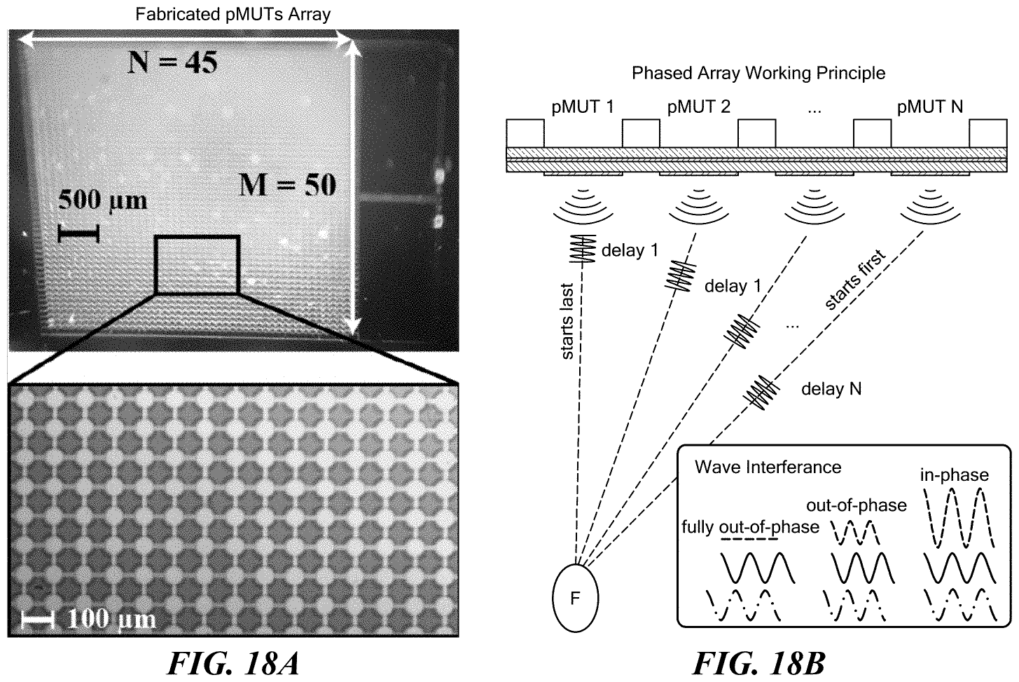

The SPL level of combined arrays can be increased and focused, for example, by using two or more pMUT arrays, each as individual ultrasonic antennas to deliver the phased acoustics. An example is shown in FIG. 16A, which shows four pMUT arrays focusing ultrasound at a focal point in a scanning area. An enlarged view of one of the fabricated pMUT arrays is shown in FIG. 18A.

Referring to FIG. 16A, each array of pMUTs can include the pMUTs connected in a parallel configuration. Each pMUT array can be treated as a single element to provide several advantages. For example, for the 4 arrays shown in FIG. 16A, the reduction of channels needed to control the phase of each array element (only four channels) is an advantage compared to controlling each pMUT in the array (N×M channels, for example FIG. 11C). The complexity of the electronics can be reduced by connecting each of the pMUTs in each array in parallel and using a pMUT array as depicted in FIG. 16A. Example electronics are shown in FIG. 16B, which shows a STMicroelectronics pulser board. Another example advantage is the available output power is the combined power of the four high-density pMUT arrays. The individual elements in one single pMUT array can be closely spaced (e.g., 150 μm pitch) which can limit the in-plane focusing range and require high accuracy phase shifting (e.g., <1 ns), which requires more demanding electronics. An advantage (FIG. 16A) of phase-shifting larger arrays and placing them at a larger distance between their centers (e.g., about 25 mm), is it requires less phase shifting accuracy (>100 ns) and provides more in-plane focusing range. The focal point of the whole array of arrays can be changed to focus hyperthermia treatment at an effective location. The distance between each array in a whole array of arrays can be fixed or changeable. For example, the distance between each array can less than about 1 mm, less than about 5 mm, less than about 10 mm, or less than about 25 mm. Using more complex electronic circuits and control, the focus of each pMUT in each array can be applied to a small treatment area.

Two application categories include ultrasound hyperthermia and focused ultrasound surgery or ultrasound ablation. The first category is a long but reversible therapy (about 30 min-60 min), increasing the temperature up to about 41° C. to 45° C. The second one is a short (about 30 sec) but high intensity focused ultrasound procedure that brings the tissue up to greater than about 50-60° C., creating permanent biological change. The technology can be applied to both categories.

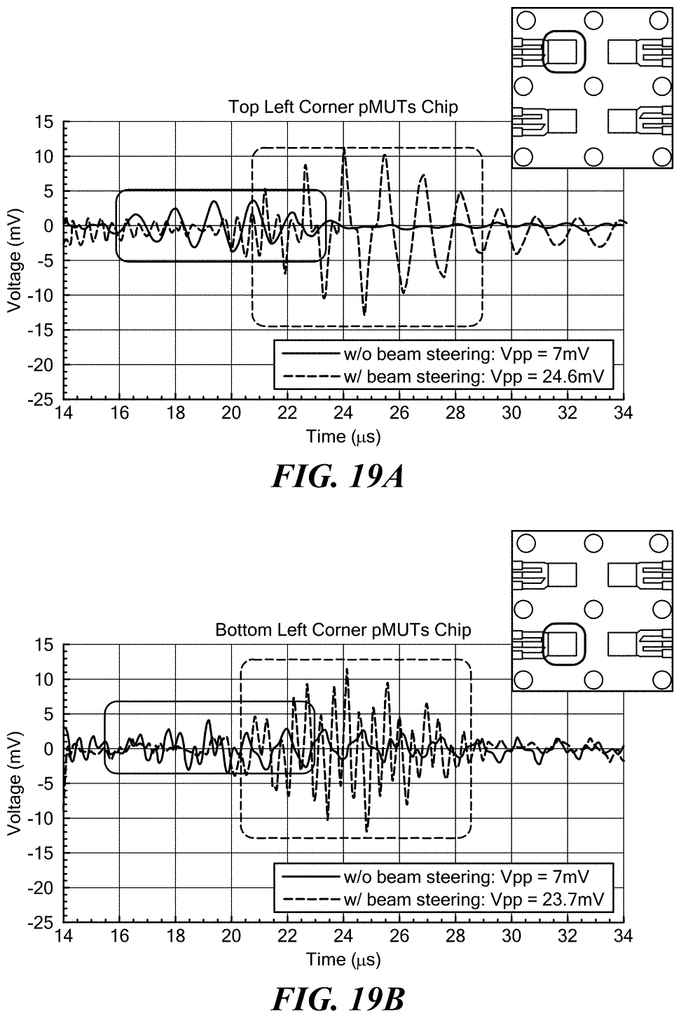

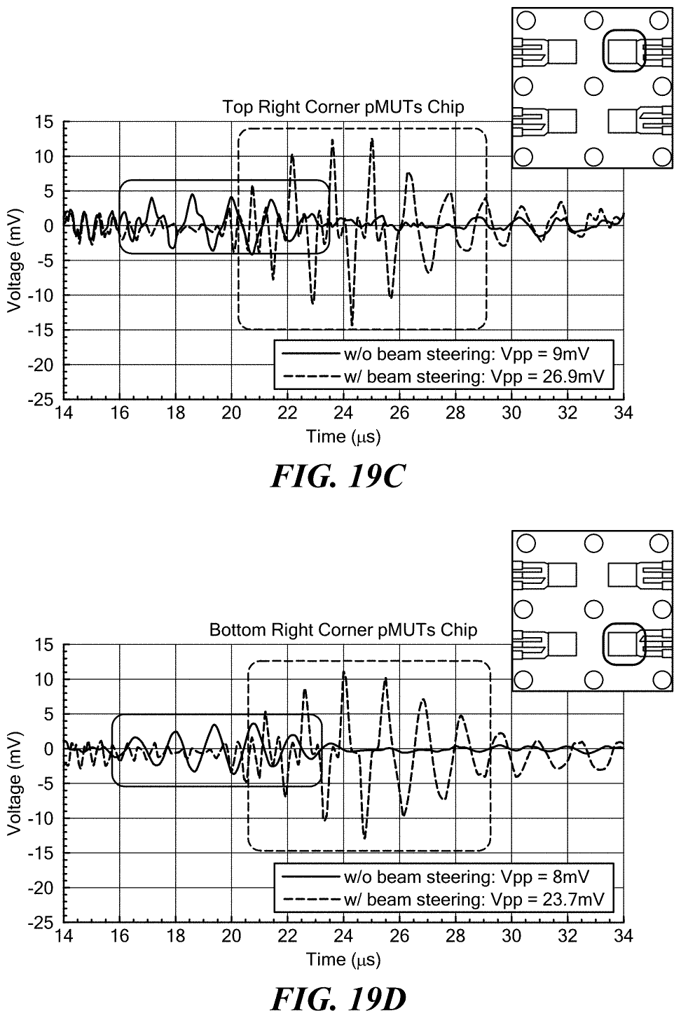

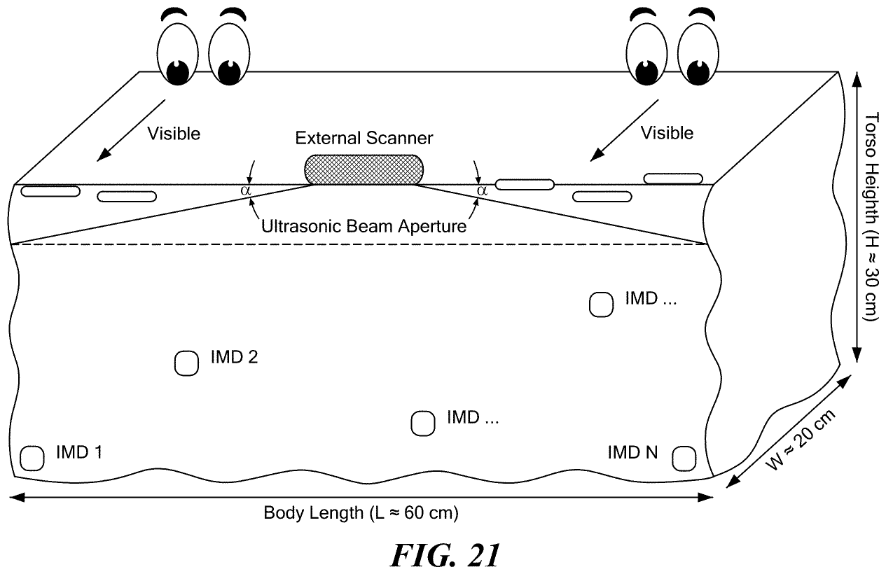

The technology can provide several advantages, for example, low form factor and biocompatible materials, thus implantable or ingestible in a body. Referring to the right of FIG. 1, when a pMUT array is implanted in a body, time delays can be utilized to focus the hyperthermia at disease areas, for example, cancer cells. The time delays can be applied to a single pMUT array as shown in FIG. 12A and FIG. 12B to deliver increased phased array SPL as shown in FIG. 14D. The time delays can be applied to arrays of pMUTs as shown in FIGS. 19A-19D to provide increased (and focusable) SPL. FIGS. 19A-19D show acoustic signal measurements on a receiver placed 5 cm from the transmitter for 4 of the pMUT chips shown in FIG. 18A. Measurements were taken both with and without the use of a beam steering, phased array technique. In FIG. 19A, the top left corner pMUT array is circled, and Vₚₚ measured 5 cm above the top left without beam steering is 7 mV, while Vₚₚ with beam steering is 24.6 mV. For all of FIGS. 19A-19D, the received voltage has an improvement from an average of 7-9 to 24-27 mV with phased arrays which allows lower power levels at the transmitter side and better sensitivity on the receiving side. Based on the sensitivity of the hydrophone, these values are equivalent to SPL_{nonfocused}=154-157 [dB] and SPL_{focused}=165-167 [dB], counting for an improvement of about 10-11 [dB]. The focusing ability can be used to scan areas as depicted in the scanning area of FIG. 16A. An aperture of focusing ability is depicted in FIG. 21 and discussed further in Example 4 below.

The array capability can be used to increase heating level, with real-time focal point reconfiguration. The reconfigurable focal point can allow the devices to be used in real time. The devices can be configured to receive instructions from outside the body by creating an acoustic communication link. There is increased interest in medical devices that can continuously monitor patients and give medical doctors useful data to improve healthcare. Enabling IMDs to communicate wirelessly with external devices through ultrasound communication links generated by pMUT arrays can be accomplished by utilizing a pMUT array, for example, as a receiver, transmitter, transducer, or a combination thereof. The pMUT devices can be miniaturized, are implantable and can reach deeper signal penetration as compared to common HIFU or radio frequency communication techniques, while maintaining a signal intensity below 720 mW/cm², which is the limit imposed by the Food and Drug Administration (FDA).

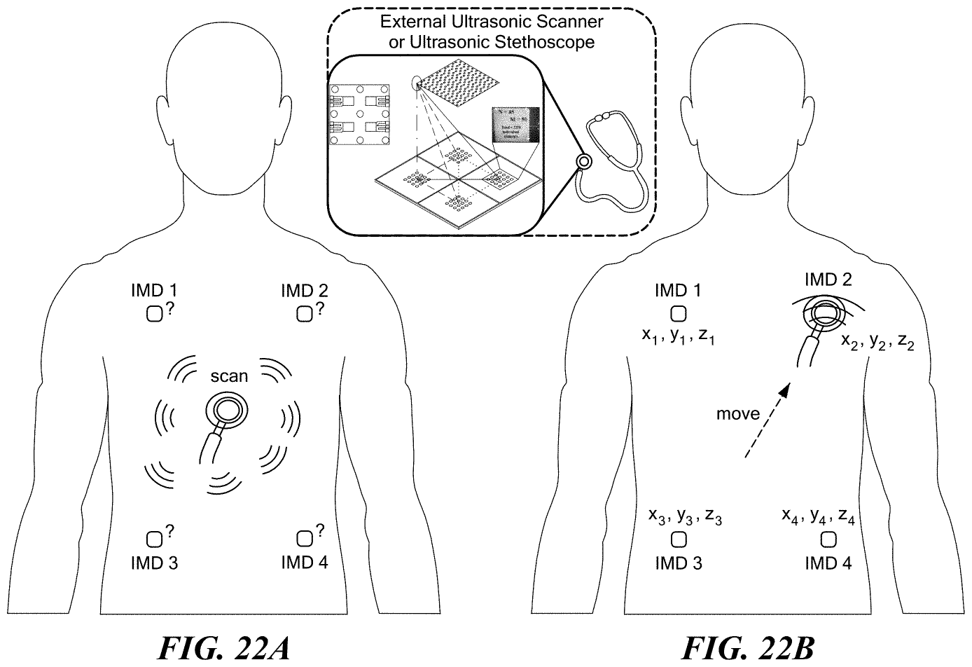

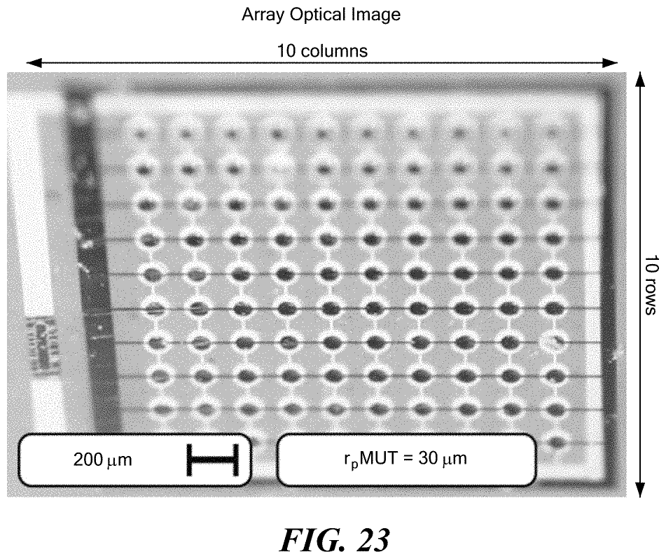

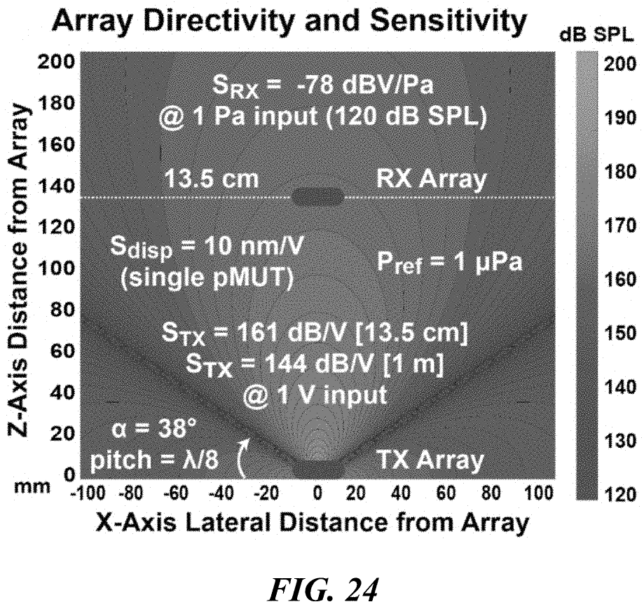

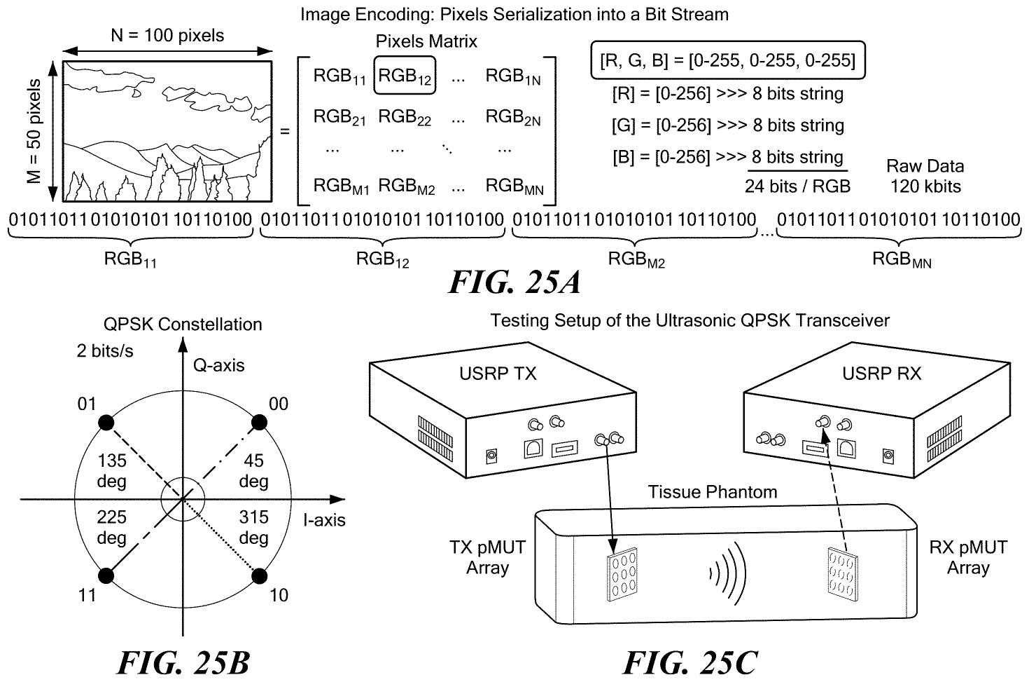

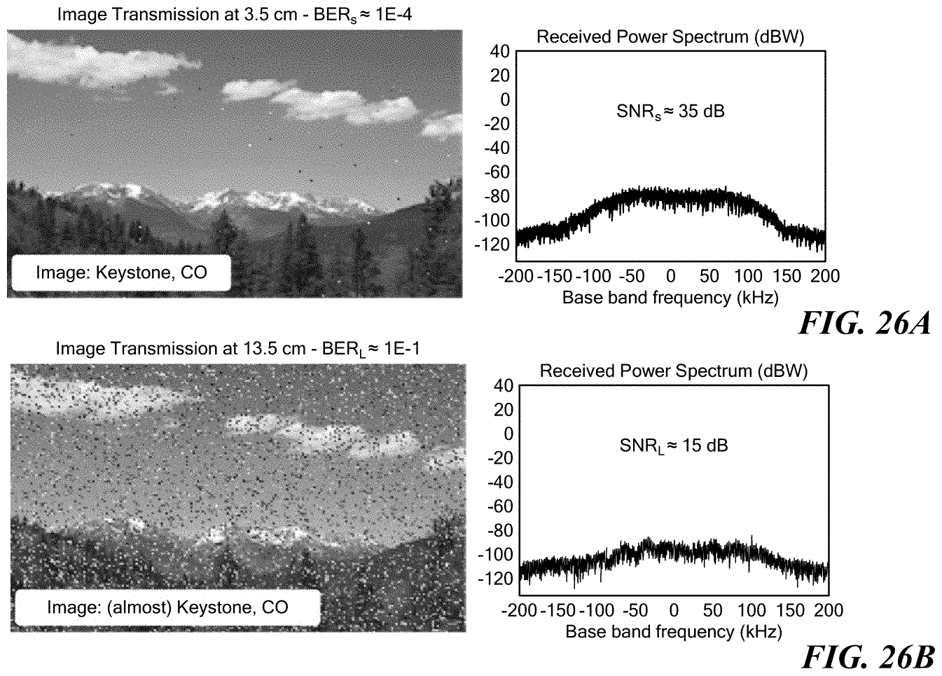

The pMUT array technology can be utilized to detect implanted medical devices having various sizes (Example 4). Referring to FIG. 21, implantable medical devices (IMD 1, IMD 2, IMD N, . . . ) are not externally visible to the eyes. One or more pMUT arrays can be utilized as an external or internal scanner to locate IMDs. FIGS. 22A-22B show an example of a planned scanning protocol using an external ultrasonic scanner or ultrasonic stethoscope. At the end of the protocol in FIG. 22A, all the positions of the devices have been detected and the stethoscope can be moved on top of the IMD of interest to improve the ultrasonic communication link (both for data and power transfer). FIG. 22A illustrates a first scan central to the body torso to find the position of the IMDs. FIG. 22B illustrates once the IMDs have been found, an ultrasonic stethoscope can be moved on top of the device (i.e. IMD 2) of interest to communicate with. An example of a pMUT array utilized for an ultrasound communication link (transmit and receive) is shown in FIG. 23. In FIG. 24, the transmission sensitivity of the array is calculated, which is the SPL at a certain distance given an input signal of 1V. Similarly, the receiving sensitivity of the array is calculated, which is the received voltage (in dBV, V_{ref}=1 V) when applying a reference input pressure level of 1 Pa (SPL=120 dB and P_{ref}=1 μPa in water or tissue), resulting in S_{RX}=−78 dBV. A 50×100 pixel image, which is shown at the left of FIG. 25A is transmitted through a tissue phantom using one pMUT array for transmission and another for receiving (FIG. 25C). Image transmission (through 3.5 cm) was accomplished with a raw bit error rate (BER) of about 1E-4 as is illustrated in FIG. 26A. The communication link can provide a fully passive implantable solution with a small form-factor that will enable on-demand sensing and communication with IMDs. Examples of acoustic communication links are presented in Example 4 and in Example 5.

The technology can be used for a variety of applications and in a variety of ways, such as heating of tissue or organ parts; ablation of tissue or organ parts; as an acoustic communication link for external commands and feedback data; real-time monitoring and ultrasonic scan of tissue or organ part that has been heated or ablated; an implantable ultrasonic platform for tissue heating and ablation; in or with other high-intensity focused ultrasound (HIFU) technologies; implantable ultrasonic platform for real-time monitoring of vital signs by establishing an ultrasonic communication link. The technology can offer a performance advantage in terms of precision of the tissue heating/ablation because the device can be implanted into the body, as compared to prior HIFU technologies that act from outside the body. The technology can be used for heating and ablation with pMUTs with focused and unfocused ultrasonic beams. The technology is can be implemented at low cost, for example, by utilizing the low-cost electronics and fabrication disclosed herein. A flexible and implantable support can be utilized instead of the PCB shown in the examples herein. The devices and systems can be flexible within a moving subject. The devices and systems can be implanted for long periods of time within a subject.

A system for ultrasonically heating biological tissue can include an implantable or ingestible device including an array of pMUTs supported on a substrate, and the array can be in communication with a controller to control the array to focus ultrasonic transmissions at biological tissue in the body to heat the biological tissue. The system can be configured wherein the array of pMUTs includes a piezoelectric layer activatable by application of a voltage to a pair of electrodes.

The system can include a power source. For example, the power source can be an ultra-sonic transducer operable to convert received ultrasound to electrical power. The ultra-sonic transducer can include an array of pMUTs. Ultrasound can be transmitted to the transducer from an extra-body source, for example, to charge a battery, capacitor, or power storage within the system.

The system can be configured wherein each of the pMUTs is in communication with a controller operative to change the focal point of the array by providing a time delay to an AC voltage applied to each of the pMUTs. The system can be configured wherein two or more arrays are provided and each array is configured with the pMUTs connected in a parallel configuration. In another example, the system can be configured wherein each of the pMUTs is in communication with a controller operative to change the focal point of the array by providing a time delay to an AC voltage applied to each of the pMUTs, and the system can be configured wherein two or more arrays are provided, wherein each of the pMUTs is in communication with a controller operative to change the focal point of the array by providing a time delay to an AC voltage applied to each of the pMUTs. The controller can be implantable or ingestible.

The system can be in communication with a sensing device, for example, a temperature sensor, a location sensor, a motion sensor, a gyroscope, an accelerometer, a cardiac rhythm monitor, a heart rate monitor, a pulse monitor, a blood pressure monitor, a glucose sensor, a drug pump monitor, a sleep sensor, a still camera, a video camera, an infrared sensor, a sensor for one or more biomolecules, a sensor for one or more pharmaceutical agents or pharmaceutical formulation ingredients, a sensor for a dissolved gas or ion, a sensor for pH, a sensor for ionic strength, or a sensor for osmolality. Nanoparticles can be used with the methods, devices, or systems. Examples of nanoparticles are nanoparticles including gold, silver, carbon, copper, iron, ceramic, polymer, biomolecules, lipids, quantum dots, sensing agents, targeting agents, delivery agents, chemotherapy agents, titanium, zinc, cerium, and thallium.

The system can be used for hyperthermia or for a method of tissue ablation. For example, the system or methods herein can be used to treat biological tissue including neck cancer cells, brain cancer cells, thyroid cancer cells, breast cancer cells, prostate cancer cells, kidney cancer cells, endometrial cancer cells, pancreatic cancer cells, lung cancer cells, esophageal cancer cells, bladder cancer cells, rectal cancer cells, cervical cancer cells, ovarian cancer cells, peritoneal cancer cells, sarcoma cancer cells, neuroblastoma cancer cells, leukemia cancer cells, or melanoma cancer cells. In another example, the system or methods herein can be used to treat or to mitigate fungal, bacterial, or viral infections. Treating can involve combination therapy with, for example, an antibiotic, an antifungal, or an antiviral agent.

The system can include an imaging system operative to locate cancerous or diseased cells. The imaging system can include memory, software, and processor, in communication with a controller operative to change the focal point of the array. The technology can be utilized in combination with one or more other therapies, for example, radiation therapy, immunotherapy, targeted drug therapy, chemotherapy, radiofrequency therapy, and hormone therapy.

The system can be configured to operate in real time and in response to one or more feedback loops. The one or more feedback loops can be utilized, for example, for the controller to change the focal point of one or more arrays.

The methods described herein can be implemented in any suitable computing system.

The computing system can be implemented as or can include a computer device that includes a combination of hardware, software, and firmware that allows the computing device to run an applications layer or otherwise perform various processing tasks. Computing devices can include without limitation personal computers, workstations, servers, laptop computers, tablet computers, mobile devices, wireless devices, smartphones, wearable devices, embedded devices, microprocessor-based devices, microcontroller-based devices, programmable consumer electronics, mini-computers, main frame computers, and the like and combinations thereof.

Processing tasks can be carried out by one or more processors. Various types of processing technology can be used including a single processor or multiple processors, a central processing unit (CPU), multicore processors, parallel processors, or distributed processors. Additional specialized processing resources such as graphics (e.g., a graphics processing unit or GPU), video, multimedia, or mathematical processing capabilities can be provided to perform certain processing tasks. Processing tasks can be implemented with computer-executable instructions, such as application programs or other program modules, executed by the computing device. Application programs and program modules can include routines, subroutines, programs, scripts, drivers, objects, components, data structures, and the like that perform particular tasks or operate on data.

Processors can include one or more logic devices, such as small-scale integrated circuits, programmable logic arrays, programmable logic devices, masked-programmed gate arrays, field programmable gate arrays (FPGAs), application specific integrated circuits (ASICs), and complex programmable logic devices (CPLDs). Logic devices can include, without limitation, arithmetic logic blocks and operators, registers, finite state machines, multiplexers, accumulators, comparators, counters, look-up tables, gates, latches, flip-flops, input and output ports, carry in and carry out ports, and parity generators, and interconnection resources for logic blocks, logic units and logic cells.

The computing device includes memory or storage, which can be accessed by a system bus or in any other manner. Memory can store control logic, instructions, and/or data. Memory can include transitory memory, such as cache memory, random access memory (RAM), static random-access memory (SRAM), main memory, dynamic random-access memory (DRAM), block random access memory (BRAM), and memristor memory cells. Memory can include storage for firmware or microcode, such as programmable read only memory (PROM) and erasable programmable read only memory (EPROM). Memory can include non-transitory or nonvolatile or persistent memory such as read only memory (ROM), one-time programmable non-volatile memory (OTPNVM), hard disk drives, optical storage devices, compact disc drives, flash drives, floppy disk drives, magnetic tape drives, memory chips, and memristor memory cells. Non-transitory memory can be provided on a removable storage device. A computer-readable medium can include any physical medium that is capable of encoding instructions and/or storing data that can be subsequently used by a processor to implement embodiments of the systems and methods described herein. Physical media can include floppy discs, optical discs, CDs, mini-CDs, DVDs, HD-DVDs, Blu-ray discs, hard drives, tape drives, flash memory, or memory chips. Any other type of tangible, non-transitory storage that can provide instructions and/or data to a processor can be used in the systems and methods described herein.

The computing device can include one or more input/output interfaces for connecting input and output devices to various other components of the computing device. Input and output devices can include, without limitation, keyboards, mice, joysticks, microphones, cameras, webcams, displays, touchscreens, monitors, scanners, speakers, and printers. Interfaces can include universal serial bus (USB) ports, serial ports, parallel ports, game ports, and the like.

The computing device can access a network over a network connection that provides the computing device with telecommunications capabilities Network connection enables the computing device to communicate and interact with any combination of remote devices, remote networks, and remote entities via a communications link. The communications link can be any type of communication link including without limitation a wired or wireless link. For example, the network connection can allow the computing device to communicate with remote devices over a network which can be a wired and/or a wireless network, and which can include any combination of intranet, local area networks (LANs), enterprise-wide networks, medium area networks, wide area networks (WANS), virtual private networks (VPNs), the Internet, cellular networks, and the like. Control logic and/or data can be transmitted to and from the computing device via the network connection. The network connection can include a modem, a network interface (such as an Ethernet card), a communication port, a PCMCIA slot and card, or the like to enable transmission to and receipt of data via the communications link. A transceiver can include one or more devices that both transmit and receive signals, whether sharing common circuitry, housing, or a circuit boards, or whether distributed over separated circuitry, housings, or circuit boards, and can include a transmitter-receiver.

The computing device can include a browser and a display that allow a user to browse and view pages or other content served by a web server over the communications link A web server, sever, and database can be located at the same or at different locations and can be part of the same computing device, different computing devices, or distributed across a network. A data center can be located at a remote location and accessed by the computing device over a network. The computer system can include architecture distributed over one or more networks, such as, for example, a cloud computing architecture. Cloud computing includes without limitation distributed network architectures for providing, for example, software as a service (SaaS).

### EXAMPLES

### Example 1: COMSOL Multiphysics Simulation

In order to better simulate a scenario for an ultrasonic heating system, COMSOL Multiphysics® was chosen given its capability of integrating multiple physical domains. For the pMUT simulation, the piezoelectric module was used, which coupled the electric actuation to the mechanical vibration of the membrane. An acoustic domain was necessary for the coupling between the membrane vibration and the generation of ultrasonic waves. At this point, the ultrasonic wave interaction was coupled with the targeted object to heat (in this case a thermocouple probe); thus, the bio-heating module was used.

The results of the ultrasonic and heating simulation are shown in FIG. 2, which shows a Finite Element Analysis (FEA) simulation in COMSOL Multiphysics® of the ultrasonic response of a pMUT and the relative temperature response of a probe placed at the focal distance. The graph shows the temperature increase and decrease (scaled to the number of pMUTs used in the experiment) at different operation frequencies. The pMUT was driven at three different frequencies and the heating curves of the thermocouple probe are shown in the graph. In the first period the pMUT was actuated; thus, the heating was ON, and an increase in temperature was observed, starting from ambient temperature of 20.5° C. and increasing to 21.5° C., 23° C. and 24.5° C. respectively. At this point, once the pMUT array was deactivated, heating was OFF, the temperature was decreasing exponentially.

### Example 2: Experimental pMUT Implementation and Results

In one experimental implementation, a 5×10 elements array was wirebonded to a circuit board and submerged in a deionized water tank, mimicking the human tissue properties and isolating possible electrical conduction paths (FIG. 3). FIG. 3 shows the experimental setup for ultrasonic response measurement and ultrasonic heating response in a de-ionized water tank mimicking the human tissue properties. The pMUT array of 5×10 elements is wired-bonded to a circuit board. The array is driven with a burst signal of N=10 sine waves. The acoustic pressure is measured with a Teledyne hydrophone and the temperature is measured with two thermocouple probes, one as reference (misaligned with the chip) and one for the actual temperature (aligned with the chip). The pMUTs were driven with a 2Vₚₚ burst signal of ten sinewave cycles. Firstly, the ultrasonic response was measured with a commercial Teledyne hydrophone (FIG. 4). FIG. 4 shows the ultrasonic response of the pMUT array at 700 kHz and at 2 MHz. The array is driven with a Vₚₚ=2V burst signal of N=10 cycles. The received tone is measured at a distance of D=5 cm with a Teledyne hydrophone. The ultrasonic signal has an amplitude of around 54 mVₚₚ at 700 kHz and an amplitude of around 104 mVₚₚ at 2 MHz. Secondly, the temperature was measured with two thermocouple probes (FIG. 5-6). One thermocouple probe was aligned with the chip (Device A, FIG. 5) to monitor the heating of the probe, while the second probe was misaligned (Device B, FIG. 6) and acts as reference. The pMUT elements were actuated in parallel and the probe tip placed at the focal distance of the array in order to maximize the acoustic energy. Given the biocompatibility and miniaturization capability of the pMUT arrays, this system can be employed as a continuous micro-therapy system. FIG. 5 shows experimental results of the ultrasonic heating at different frequencies: 700 kHz, 1 MHz and 2 MHz (Device A). The results show good agreement with COMSOL simulation (FIG. 2). In particular the maximum temperature increase from medium temperature (20.5° C. measured by the reference probe) are 3.5° C., 4° C. and 4.5° C. respectively to the actuation frequency of the pMUT array.

FIG. 6 shows experimental results of the ultrasonic heating at different frequencies: 700 kHz, 1 MHz and 2 MHz (Device B). This pMUT array showed slightly lower performance compared to Device A due to the misalignment of the measuring probe. Once the driving signal is switched on, there is a latency for the heating of 2-4 seconds. After 10 seconds the temperature saturates. At 20 seconds the ultrasonic signal is turned off and the temperature decays according to the equivalent thermal loss coefficient of the probe and propagation medium (de-ionized water).

The ultrasonic response of the pMUT array was measured with a commercial Teledyne hydrophone at a distance of five centimeters. The driving signal of the pMUT array and the received signal on the hydrophone are shown in FIG. 4. The pMUT was driven with a burst signal of ten sine waves. The received signal was delayed based on the propagation speed of ultrasonic waves in the medium, which was approximately 1450 meters per seconds in DI water. The received SPL was converted into voltage and acquired with an oscilloscope. The pMUT was driven at 700 kHz (out of resonance) and 2 MHz (at resonance) and the received voltages were 54 mVₚₚ and 104 mVₚₚ respectively.

The ultrasonic response of the pMUT array was measured at 700 kHz and 2 MHz at a 5 cm distance, resulting in a received voltage of 54 mVₚₚ and 104 mVₚₚ respectively (FIG. 4). Following, the heating results of the probes were collected over time at multiple frequencies, showing good matching with simulation results (FIG. 2). When the pMUTs signal was switched ON, the temperature rose after a latency of two to four seconds and saturated after ten seconds. When the driving signal was switched OFF, the temperature decayed exponentially according to the equivalent thermal loss coefficient of the probe and medium properties. In this demonstration, the ultrasonic heating increased the relative temperature up to 4° C. starting from 20.5° as shown in FIG. 5 (Device A) and 6 (Device B). For the Device A, the maximum temperature increments from medium temperature were 3.5° C., 4° C. and 4.5° C. respectively to the actuation frequency of the pMUT array. On the other hand, for the device B, the pMUT array showed slightly lower performance compared to Device A due to the misalignment of the measuring probe. In particular, the maximum temperature increments from medium temperature were 2° C., 3° C. and 3.5° C. respectively to the actuation frequency of the pMUT array. When applied to a human tissue, that has an average temperature of 37° C., this can result into a local heating of the tissue of up to 41° C. These results support the development of implantable pMUT-based bio-heating platforms for hyperthermia micro-therapies and continuous monitoring based on intrabody communication links.

### Example 3: Phased-Array Platform Based on Directly Modulated Ultrasonic Transducers

In this example the starting system of a phased-array platform was a single DMUT circuit shown in FIG. 10. The system allows to directly feed an ON/OFF keying signal into the transducer and at the same time boost the voltage on top of it, improving the output pressure and SNR. The focus was to exploit the DMUT circuit for its voltage boosting capability and take advantage of the low input signal. In particular, the circuit could be driven with signals of less than about 3.5V, which allowed for its integration with commercial micro-controllers. This allowed piloting multiple DMUT circuits and controlling their phase-shifting (or time-delay) in order to implement a phased-array technique.

The circuit in FIG. 10 includes a Bipolar Junction Transistor (BJT) acting as a switch when driven in its cut-off region (low voltage) and saturation region (high voltage). By applying a train of pulses at the base of the transistor, this will connect and disconnect a DC biased LC tank to the acoustic transducer. When modulating the switch with an ON/OFF signal, the LC tank will abruptly change the resonance frequency due to the high capacitance of the pMUTs array. In this way, a portion of the energy stored by the inductance of the LC filter, will be stored by the transducer. This mechanism can explain the generation of the high voltages at the output of the DMUT system.

The single DMUT circuit was laid out in arrays on a PCB in order to pilot ten individual channels of a pMUT array. An example electrical diagram is shown in FIG. 11A, while the fabricated PCB and the connections to the micro-controller Teensy 3.6 to pilot the array are shown in FIG. 11B. An optical image of the fabricated pMUT array is shown in FIG. 11C. This image highlights the rows one to five (connected to ground) and the columns zero-nine (connected to the outputs zero-nine of the DMUT array).

The objective of the phased-array technique was to generate constructive interference of the ultrasonic waves at a certain focal point in space. This allows a higher acoustic signal, improves the transmission distance and the SNR. The focusing was achieved by delaying the signals of different columns of the pMUT array in order for the ultrasonic signal to arrive in phase at the desired distance from the array. By doing so, the waves add up constructively instead of creating destructive interference. The driving signals implemented in the micro-controller are shown in FIG. 12A, and the relative outputs of the DMUT array, which connect directly to the columns of the pMUT array, are shown in FIG. 12B. The DMUT array functionality was tested by creating an ultrasonic link between a 10×5 pMUT array and a commercial Teledyne hydrophone submerged in a silicone oil tank, as shown in FIG. 13. Based on its datasheet, the hydrophone has a sensitivity of S=−228 dB/V, which allowed conversion of the received voltage signal into sound pressure and comparison to mathematical model results. Furthermore, the pMUT array was wire-bonded to a PCB, the rows were connected to ground and the columns were connected to the outputs of the DMUT array respectively, allowing for the formation of 10 individual channels.

Mathematical modeling of the output pressure of the pMUT array was modeled starting from experimental measurements with a Digital Holographic Microscope. The main parameters are the peak displacement (dₚ) and resonance frequency (fₛ) of the individual elements. The output pressure at the surface of one pMUT could be expressed as following:

$$\begin{matrix}P = {v}_{p} \cdot {Z}_{a} \cdot {A}_{eff} & ( Eq . 1 ) \\ {V}_{p} = 2 π \cdot {d}_{p} \cdot {f}_{s} & ( Eq . 2 ) \\ {Z}_{a} = \frac{{p}_{0} \cdot {c}_{0}}{{A}_{eff}} & ( Eq . 3 ) \\ {A}_{eff} = \frac{2 π \cdot {a}^{2}}{3} & ( Eq . 4 )\end{matrix}$$

where vₚ is the peak membrane velocity, Zₐ is the acoustic impedance, A_{eff} is the effective area of the pMUT, a is the membrane radius, p₀ is the density of the silicone oil and c₀ is the speed of sound in the silicone oil.

When driving an entire pMUT array, the output pressure will be a function of the combination of all the ultrasonic waves based on the phase-shift (or time-delay) of the elements, the geometric spread of the acoustic waves, the directivity of the array and the medium attenuation. A closed form of the output pressure of the array could be expressed as follows:

$$\begin{matrix}{P}_{array} = \frac{P \cdot k \cdot {a}^{2}}{2 r} \cdot D \cdot {e}^{- γ \cdot r} \cdot {e}^{- i \cdot k \cdot r} \cdot φ ( t ) & ( Eq . 5 ) \\ D = \frac{48 \cdot {BesselJ}_{3} ( k \cdot a \cdot sin ( θ ) )}{{( k \cdot a \cdot sin ( θ ) )}^{3}} & ( Eq . 6 ) \\ γ = \frac{α}{20 \cdot {log}_{10} e} & ( Eq . 7 )\end{matrix}$$

where r is the distance at a certain coordinate from the array, θ is the angle formed with the array at the distance r from the array, D is the directivity, γ is the attenuation term, a is the absorption coefficient of the silicone oil, k is the wave number and Φ(t) is the phased-array delay coefficient.

When all the pMUTs are driven with the same signal (equal delays) (e.g., FIG. 14A), both constructive and destructive interference will be happening at a certain distance from the array. On the other hand, when the delays are set for each column of the array to reach a certain focal distance at the same time, the acoustic waves will add up and maximize the pressure in that region (e.g., FIG. 14B). A mathematical model of the SPL for the pMUT array, shown at the right of FIG. 13, was calculated in FIG. 14A. In FIG. 14A, the phased array technique was off. In FIG. 14A, the SPL legend is at right, with lighter shades indicating higher SPLs up to about 150 dB. In FIG. 14B, the phased array, including the timing of the driving signals was on, and a higher SPL was achieved with the phased array on. The lighter shades indicate a focused SPL up to about 166 dB. The mathematical model showed an improvement of about 16 dB SPL when applying the phased-array technique.

The DMUT array was employed to drive an array of pMUTs implementing the phased-array technique. The time-delays of each column of the array were coded in a Teensy 3.6 micro-controller. The Teensy can only supply about 3.5 V for each channel, therefore driving the pMUT array directly will result into low output pressures. Instead, the DMUT system allows to output high voltage signals while being driven by low amplitude signals supplied by the micro-controller. The measurement results are shown in FIG. 14C and FIG. 14D with the phased-array OFF and ON, respectively. The hydrophone measured a maximum amplitude peak-to-peak of V_{OFF}=5 mVₚₚ (FIG. 14C) and V_{ON}=25 mVₚₚ (FIG. 14D). Given the sensitivity of the receiver, this converts into a sound pressure of SPL_{OFF}=152 dB and SPL_{ON}=165 dB, respectively. The measurements showed an improvement of 13 dB SPL when using the phased-array technique implemented with the DMUT array, validating the mathematical model.

### Example 4: Real-Time Monitoring of Intrabody Networks Through an Acoustic Discovery Architecture

In this work, constructive interference was utilized to focus arrays of pMUTs and to implement monitoring of intrabody networks. Initially, each pMUT was modeled as an electromechanical-acoustic device that is able to convert energy from the electrical domain to the mechanical domain and then ultimately to the acoustic domain. An example working principle of a single pMUT is depicted in FIG. 9. Modeling of a particular frequency f (resonance) was determined using the following equations:

$$\begin{matrix}{f}_{air} = \frac{{3.19}^{2}}{2 {π a}^{2}} \sqrt{\frac{D}{μ}} & ( Eq . 8 ) \\ {f}_{tissue / liquid} = \frac{{f}_{air}}{\sqrt{1 + 0.34 \frac{2 pa}{μ}}} & ( Eq . 9 )\end{matrix}$$

where a is the radius of the membrane, D is the flexural rigidity of the membrane, μ is the weighed density of the membrane with respect to the film thicknesses, and ρ is the medium density (e.g., ρ=971 kg/m³ when using silicone oil). For example, for a membrane of radius a=25 μm, the resonance frequency in living tissue will be about fₜᵢₛₛᵤₑ=700 kHz.

When the membrane of the pMUT vibrates at a certain frequency, it generates acoustic waves that will propagate into the medium in which the device is placed on. This can depend on the following equations:

$$\begin{matrix}P = 2 π f \cdot {d}_{p} \cdot {Z}_{a} \cdot {A}_{eff} & ( Eq . 10 ) \\ {A}_{eff} = \frac{{π a}^{2}}{3} & ( Eq . 11 )\end{matrix}$$

where dₚ is the peak membrane displacement, Zₐ is the real part of the acoustic impedance of the medium and can be derived from Eq. 3 (Example 3) above, c₀ is the propagation speed of the acoustic waves (e.g., in this example, c₀=1350 m/s when using silicone oil), and A_{eff} is the effective area of the membrane. For a measured membrane displacement of dₚ=5 nm when 1 V of AC signal is applied at the resonance frequency, the pressure at the membrane interface is about P=28.8 kPa. This can be converted to SPL based on the following equation:

$$SPL = 20 \cdot {log}_{10} \frac{P}{{P}_{ref}} \qquad ( Eq . 12 )$$

where P_{ref}=1 μPa. This is equivalent to SPL_{surface}=210 dB at the membrane surface, which will attenuate while propagating. Depending on the medium and the frequency of operation, the acoustic waves will have different absorption coefficients, and examples are presented in Table 1.

TABLE 1
Examples of Ultrasound Wave Attenuation in Tissues.

| Material: | att. [dB/cm/MHz]: |
|---|---|
| Fat | 0.63 |
| Blood | 0.63 |
| Bone | 20.0 |
| Lung | 41.0 |
| Liver | 0.94 |
| Kidney | 1.00 |
| Brain | 1.2-2.5 |
| Gray matter | 0.5-1.0 |
| Skeletal muscle along fibers | 1.3 |
| Skeletal muscle cross fibers | 3.3 |

For example, at 700 kHz in silicone oil, the absorption can be about 0.1 dB/cm. By considering this coefficient and the radial geometric spreading at 5 cm from the surface of the pMUT, the pressure can go down to about SPL_{5 cm}=115 dB (5 cm).

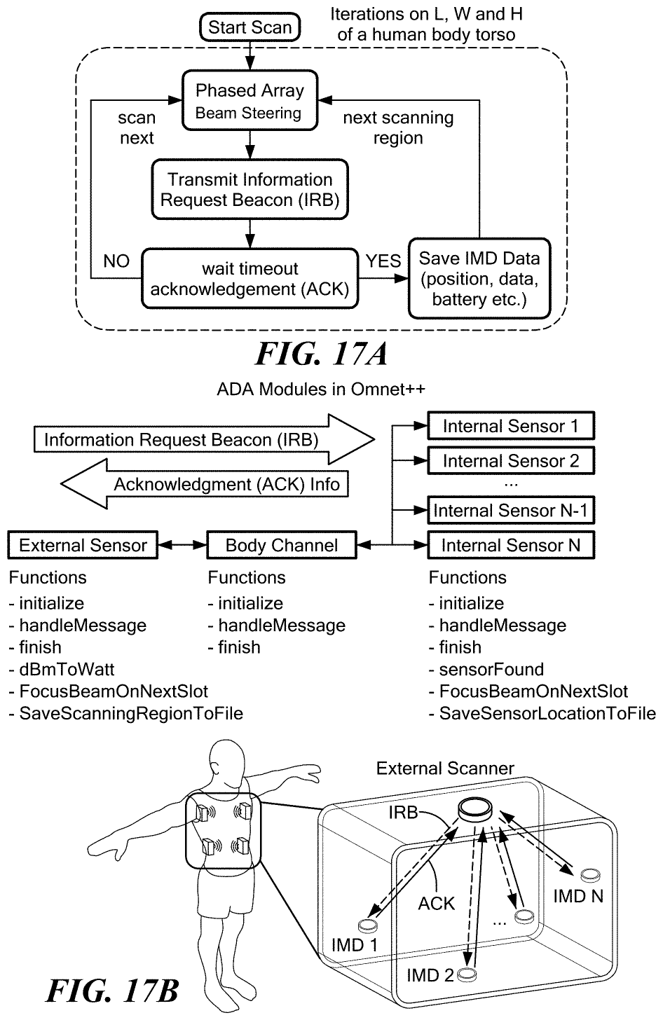

The concept (FIG. 15A) of an acoustic discovery architecture (ADA) was investigated from the perspective that more and more IMDs will be placed in patients in the context of the Internet of Medical Things (IoMT). Examples of IMDs can be heart defibrillators (pacemakers), insulin pumps, pH sensors, thermal measurement devices, imaging devices, and hyperthermia treatment devices. Each IMD can have its own information stored locally. This can be, for example, the vital signs that are being collected, the coordinates of the device's location, the battery charge level, date of implantation, and measured conditions. To this purpose, ADA was designed as an algorithm for intrabody networks (INs) created with the purpose of finding IMDs. This was achieved by scanning a body area with ultrasonic communication links and generating a map with information such as location, battery status (if any), and up-link sensing data from the IMDs. The ultrasonic communication links were generated through the use of the pMUT arrays, which are biocompatible, CMOS-compatible, and of low power consumption. An example ADA working principle with two IMDs, labeled in communication with pMUT arrays, is shown in FIG. 15B. For example, an EAT (external scanner, FIG. 15A) scanner system could be placed on top of a body torso and a phased array steering technique can focus acoustic energy on different regions of the body and send information request beacons (IRB s). If a device exists in that same region, it will respond with an acknowledgement (ACK) beacon back to the external scanner. It can be assumed that the IMDs are always in an IDLE state for power saving, and that they are woken up only in the presence of an external acoustic wake up signal. As illustrated in FIG. 17A, transmission of an information request beacon (IRB) could be used as a wake-up signal.

A single pMUT could only generate enough power to communicate in a subcentimeter range. Therefore, in order to increase the communication range, there was a need for converting more energy into the acoustic domain.

The pMUT fabrication (e.g., FIG. 8) was performed on a double-side polished silicon wafer (DSP-Si) of 300 μm to facilitate the fabrication process on both sides. The structural layer, 500 nm of silicon oxide (SiO₂), was then deposited at low temperature through plasma-enhanced chemical vapor deposition (PECVD). The bottom electrode, 100 nm of platinum (Pt) was deposited via e-beam evaporator. An additional adhesion layer of 5 nm of titanium (Ti) was required in between the electrode and the oxide. At this point 700 nm of aluminum nitride (AlN) piezoelectric layer was reactive sputtered. The AlN layer was patterned with photoresist (PR) and etched via hot phosphoric acid at 85° C. in order to create electrical access vias to the bottom electrode. For the top electrode patterning, the lift-off technique was used. First the PR was patterned and then 150 nm of gold (Au) layer deposited. In the end, the wafer was sonicated to remove the leftover gold. The last step was the release of the membrane. A hard mask layer was used to pattern the cavities (SiO₂) of the pMUTs with back side alignment, and the silicon under the membrane was then etched via deep reactive-ion etching (DRIE).

AlN was chosen as the piezoelectric material for its low dielectric losses and biocompatibility. The AlN could be further optimized by doping it with scandium (ScAlN), which can improve the electromechanical coupling (k²ₜ). This can result in an increased output pressure at the surface of a pMUT membrane and the receiving sensitivity. Moreover, sputtered lead zirconate titanate (PZT) had been explored, which results in a higher kₜ², but the drawback is that the lead is not biocompatible, and it requires an additional packaging for implantable medical devices applications.

After fabrication, the microfabrication yield was evaluated at the chip level. Each pMUT array was designed to fit in an 8×8 mm² die. In this work, the array contained 45 rows and 50 columns, for a total of 2250 elements (FIG. 18A). Even though some elements of the array might have resulted in a broken membrane during the last fabrication step (DRIE), the chip was evaluated for its overall SPL captured with a commercial hydrophone at a fixed distance. This allowed determination if an array can be used to build the four-array beam-forming boards (e.g., FIG. 16A, FIGS. 19A-19D), based on the maximum distance desired in an ultrasonic communication link. Assuming a threshold of SPL_{measured}>SPL_{theoretical}−6 dB, the chip level yield results to Yield_{chip/array}>80%. At this point, once the chips had passed this first yield test, these were mounted on the printed circuit board (PCB) with carbon tape and wire-bonded to aluminum pads. Potential failures at this level included breaking the chip while trying to adjust on the carbon tape and scratch-off the gold wire-bond pads from the chip when repeating the wire-bonding procedure. For this reason, once the wire-bonds were successfully done, they were sealed locally with a silicone gun in order to avoid breaking them when submerging the chip in the silicon oil tank (or taking them out). Finally, the system level yield could be estimated to be Yield_{system}>90%. Finally, the overall yield can be approximated as Yieldₜₒₜₐₗ=Yield_{chip/array}·Yield_{system}>72%.

For this reason, the pMUTs were fabricated into the larger arrays (FIG. 18A) to harness their combined power. When two different acoustic waves, originating from neighboring pMUTs, travel in the medium and interact with each other at a certain point in space, they will suffer from destructive interference (similar to the optical domain) if they are out-of-phase. To mitigate this effect, the technique of phased arrays was used to make all of the output acoustic waves from individual pMUTs to arrive in phase at a certain point in space and have constructive interference (e.g., FIG. 18B).

To increase the acoustic energy further, four pMUT arrays were used as individual ultrasonic antennas to perform the phased arrays (FIG. 16A, FIGS. 19A-19D). Connecting all the pMUTs in each array in parallel and then treating the full array as a single element was found to provide several advantages. The first one was the reduction of channels needed to control the phase of each array element (only four channels) as compared to controlling each pMUT in the array (N×M channels), therefore reducing the complexity of the electronics. Example electronics are shown in FIG. 16B. The second advantage was that the available output power is the combined power of four high-density non-phased arrays. For example, the individual elements in one single array are closely spaced (150 μm pitch) which limits the in-plane focusing range and requires high accuracy phase shifting (<1 ns), which calls for more demanding electronics. An advantage (FIG. 16A) of phase-shifting the larger arrays and placing them at a larger distance between their centers (25 mm), required less phase shifting accuracy (>100 ns) and gave more in-plane focusing range. Lower cost electronics could also be utilized. Ultimately, the focal point of the whole array of arrays could be changed accordingly and a scanning algorithm could then be implemented.

The combined pressure of an array of pMUTs at the surface can be expressed as following:

P_{array}=N·M·P_{single}·\sqrt{F} (Eq. 13)

Where (N×M channels) is known, and F is the filling factor, defined as the ratio between active area and the total area:

$$F = \frac{Filled Area}{Total Area} = \frac{N \cdot M \cdot {π a}^{2}}{N \cdot M \cdot {pitch}^{2}} \qquad ( Eq . 14 )$$

At this point, each individual array could be approximated with an omnidirectional radiating element and the formula for the phased array could be applied as following:

$$\begin{matrix}{P}_{focused} = {\sum }_{i = 1}^{R} {\sum }_{j = 1}^{C} \frac{{P}_{array} \cdot k \cdot {a}^{2}}{2 \cdot {r}_{ij}} D ( {Θ}_{ij} ) {e}^{- {ikr}_{ij}} {e}^{- {γ r}_{ij}} φ ( t ) & ( Eq . 15 ) \\ D ( Θ ) = \frac{48 \cdot {J}_{3} [ ka \cdot Sin ( Θ ]}{{[ ka \cdot Sin ( Θ ) ]}^{3}} & ( Eq . 16 ) \\ D ( Θ ) = \frac{48 \cdot {J}_{3} [ ka \cdot Sin ( Θ ]}{{[ ka \cdot Sin ( Θ ) ]}^{3}} & ( Eq . 17 ) \\ γ = α \cdot {log}_{10} ( e ) & ( Eq . 18 ) \\ k = \frac{2 π \cdot f}{c} & ( Eq . 19 )\end{matrix}$$

where R and C are the rows and the columns of the external scanner, which in this case is a 2×2 array of pMUT chips, and rᵢⱼ is the radial distance of a point in space (in this case the focal point) from a pMUT array defined with the sum indices i and j. The acoustic waves will decay with the inverse low 1/rᵢⱼ, which is due to the geometric spreading of the ultrasounds on a sphere. Furthermore, there are the following functions: D(Θ) is the array directivity, defined as a function of the third-order Bessel function J₃, γ is the medium absorption function, and α is the medium absorption coefficient (dB/m). The e^{-ikrij} term instead represents the exponential-form of a traveling acoustic wave. The function, Φ(t), is the phased array percentage term that take into consideration the amount of interference based on the delays of each array. When the delays are adjusted accordingly, this function is equal to 100%, allowing maximum constructive interference.

Based on the formulas and the design parameters of the pMUT array, the non-focused SPL_{NF}=155 dB and focused SPL_{F}=168 dB at 5 cm from the four-array PCB, was then to be studied empirically. FIGS. 19A-19D show acoustic signal measurements on a receiver placed 5 cm from the transmitter for 4 of the pMUT chips configured as in FIG. 18A. Measurements were taken both with and without the use of a beam steering, phased array technique. For example in FIG. 19C, the top right corner pMUTs array is circled, and Vpp measured 5 cm above the top right without beam steering is 9 mV, while Vₚₚ with beam steering is 26.9 mV. Considering the measurements of FIGS. 19A-19D, the received voltage had an improvement from an average of 7-9 to 24-27 mV with phased arrays, which allowed lower power levels at the transmitter side and better sensitivity on the receiving side. Based on the sensitivity of the hydrophone, these values are equivalent to SPL_{non-focused}=154-157 [dB] and SPL_{focused}=165-167 [dB], counting for an improvement of about 10-11 [dB]. The focusing ability can be used to scan areas as depicted in the scanning area of FIG. 16A. An aperture of focusing ability is depicted in FIG. 21 and discussed further in FIG. 24 below. This SPL levels correspond to an absolute peak pressure of respectively P_{peak-NF}=56 Pa and P_{peak-F}=177 Pa, both operating at the center frequency of the array fₛ=700 kHz. At this point it was important to compute the MI which is a parameter that defines the bio-effects of an ultrasound beam on the human tissue (mechanical stress or damage). Assuming the definition of MI as:

$$MI = \frac{{P}_{peak}}{\sqrt{{f}_{s}}} \qquad ( Eq . 20 )$$

the MI_{NF}=0.067 and the MI_{F}=0.211; both resulted to be well below the limit set by the FDA, which is MI_{FDA}=1.900.

In order to implement the ADA in a network simulator, there was the need to collect experimental data from the communication links. These data included ultrasonic transducer sensitivity, data loss from the medium, power consumption, and propagation delays. For this purpose, an experimental setup consisting of an ultrasonic transmitter, a tank filled in with silicone oil, and an ultrasonic receiver was prepared as shown in FIG. 16C.

The transmitter was meant to function as a scanner by performing the phased array technique on the pMUT chips. The first scanner prototype demonstrated was designed on a PCB on which four 8-mm² pMUT chips are placed at the vertices of a d=25 mm² square (FIG. 16A). Each chip contains a rectangular lattice array of pMUTs, N=45 columns and M=50 rows (e.g., FIG. 18A). All the 2250 elements of a single chip were connected in parallel and driven together. Each of the chips was then driven with a different signal, counting four channels in total. The signals were generated with a STMicroelectonics STEVAL-IME011V2 microcontroller pulse board shown in FIG. 16B. The output signals of the microcontroller are reconfigurable and the delays/phases are adjusted in order to steer the beam of the array at the desired location in space and focus the acoustic energy. An example of the principle is shown in FIG. 18B.

In this experiment, both the transmitter and receiver were submerged into the tank (FIG. 16C). In real-life applications, the transmitter could be configured a scanner that is external to the body and is not necessarily subject to miniaturization. The receiver was a commercial Teledyne TC4038 hydrophone that emulates the properties of an IMD receiver. In Example 5 below, the use of pMUT arrays as receivers was further studied. Using the hydrophone, the main parameter of interest was the sensitivity of the hydrophone, since this will set the maximum needed power from the transmitter in order to establish a communication link. In order to minimize this power, an external scanner would implement the phased array technique and focus the beam on the hydrophone.

For example, F can be the point with coordinates XF, YF, and ZF at which it is desired to focus the energy and C the center of one pMUT array with coordinates XC, YC, and ZC. It is assumed that F is in the acoustic far-field relatively to C. Based on the speed of sound in the medium the travel time for each signal from C to F can be computed as following:

$$\begin{matrix}{D}_{FC} = \sqrt{{( {X}_{F} - {X}_{C} )}^{2} + {( {Y}_{F} - {Y}_{C} )}^{2} + {( {Z}_{F} - {Z}_{C} )}^{2}} & ( Eq . 21 ) \\ {T}_{FC} = \frac{{D}_{FC}}{c} & ( Eq . 22 )\end{matrix}$$

For example, if the focus of the energy (e.g., focal point in FIG. 16A or FIGS. 19A-19D) is to be at 5 cm from the PCB on top of a pMUT array, the signal delays can be the following: τ_{A}=7.5 μs, τ_{B}=3.5 μs and τ_{C}=0 μs (FIGS. 19A-19D and Table 2).

TABLE 2
Example Data-Sheet from Experimental Setup
for Network Simulation of the ADA.

| Parameter | Description | Variable | Value | Unit |
|---|---|---|---|---|
| Sensitivity | Teledyne TC4038. | S | −228 | dB/V |
| Velocity | Ultrasonic waves speed | c | 1350 | m/s |
| | in silicone oil (similar | | | |
| | to the living tissue). | | | |
| Density | Density of the silicone | ρ | 970 | kg/m³ |
| | oil (similar to the living | | | |
| | tissue). | | | |
| | | τ_{A} | 7.5 | μs |
| Delays | Time delays. | τ_{B} | 3.5 | μs |
| | | τ_{C} | 0 | μS |

OMNET++ is a discrete time network simulator based on C++ programming. Within this framework, it was possible to abstract all the physical components of a communication link into modules, which are illustrated in FIG. 17B. The three main modules of interest were: the External Sensor, which can be the phased array platform with the four pMUT chips, the Body Channel which was the communication channel formed in the tank filled with silicone oil, and in the end the multiple Internal Sensors which were all modeled based on the hydrophone receiver.

In the network simulation, it was assumed that the ultrasonic beam can reach each part of an average human torso of an estimated volume of 60×30×20 cm³. Although this is true regarding the ultrasonic beam's intensity, it was not entirely true regarding the angle. While this assumption might be optimistic, in real-life application, this consideration will only affect devices implanted very close to the surface of the torso, which is a rare case for IMDs. The kind of devices implanted at the surface level or subcutaneous level normally do not need to be found since their location can be spotted, for example, by the eye. These devices would not be hit by the ultrasonic beam based on the limit of the beam-steering angle. An angle limit of α=15° as illustrated in FIG. 21 was estimated.

Each module was assigned several parameters based on data acquired in the experimental setup (Table 2) and each implemented some standard functions: initialize and finish was in charge to start and stop an OMNET++ module while handleMessage was in charge of the communication link between different modules. Furthermore, for each module ad hoc functions were defined to model their behavior as in the experimental setup.

First of all, the External Sensor module was implemented with the following functions.

InformationRequestBeacon (IRB), which generates a beacon or message to be sent out through the communication channel in order to acquire information about the IMDs. For example, in real-life implementation, this can contain an encoded key signature in order to trigger the implanted receiver.

TimeOut in charge of counting the elapsed time after the IRB was sent out. If the transmitter receives back the ACK Info within a certain timeout, then an IMD would be registered for that position.

ReadReceivedData reads the received data from the ACK.

SaveScanningRegioToFile saves the IMDs data to file.

PhasedArrayTransmission in charge of dividing the scanning region (in this case the body torso) into scanning steps according to the scanDelta parameter, determining the number of iterations. For each iteration, different delays for the phased array technique will have to be applied.

Secondly, the Body Channel module was configured with the following functions.

ForwardMessage: This is the main function of the Body Channel that is mainly in charge of forwarding the packages from the External Sensor to the multiple Internal Sensors and the other way around. The main messages are the IRB and the ACK.

TimeDelay: It introduces a delay on sending the packages through the communication channel in order to emulate the delay based on the speed of sound in the silicone oil (or the human body). In the simulation a constant of c=1350 m/s was assumed.

RandomPackageLoss: It is in charge of adding random package losses in the communication links. Physically, this can be due to interference at different tissue interfaces, power loss during the transmission, misalignment of the phased array beam with the IMD's receiver, and so on.

The Internal Sensor module implemented the following.

WakeUp: Upon the reception of an encoded acoustic signal, the IMD will wake-up from an IDLE state to a fully functional ACTIVE state. For this to happen, the encoded signature needs to match the one of the IMD.

AcknowledgmentInfo (ACK): This is the package sent by the IMD to the external scanner as an ACK of its existence inside the body. In real life applications, this package could be transmitted broadly through the whole body or use the phased array technique to focus the energy on the external transducer. This will require a source localization technique to find the position of the scanner. To simplify the simulation, it is assumed that the ACK is sent back to the transmitter on a straight line path.

GetSensingData: Besides the position of the IMDs, it is possible to transmit other information such as the power level of the device (if it has an embedded battery) and all the acquired data by the sensors envisioned to be part of the medical device. For simplicity, only the position information was sent in the simulation in this work.

Once all the modules were programmed, the ADA algorithm illustrated in FIG. 17A was implemented. Initially, during the Start Scan phase, the volume to be scanned was meshed according to the scanDelta parameter, which defined the scanning accuracy and the number of scanning iterations to be done. At each iteration, the phased array beam steering was performed in order to focus the acoustic energy at a certain location. At this point the IRB was transmitted. If an IMD was present at that particular location and if the acoustic energy is higher than the sensitivity of the receiver, the IMD would be able to receive the IRB. At this point, assuming that the IMD has a wake-up receiver, the device would turn on from an IDLE state to a fully ACTIVE state and reply back to the receiver (ACK). The external receiver has a wait timeout until passing to the next region. If data were received before this time, it will be saved, otherwise ADA would assume that there is no IMD in that location and pass to the next scanning region.

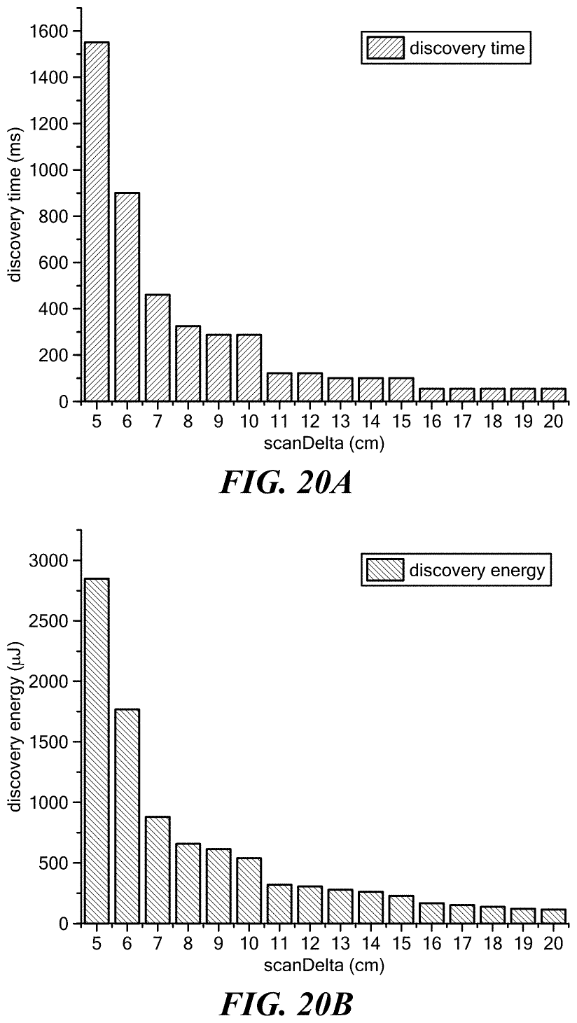

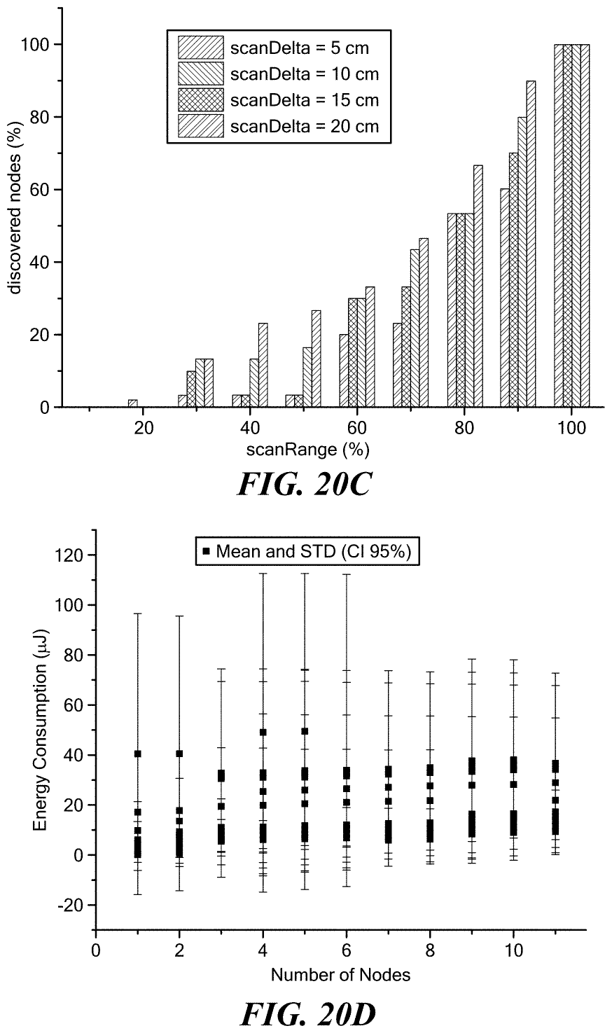

In order to start the simulations, there was the need to virtually place the implanted IMDs inside the body. In this example, ten different devices were assigned random coordinates inside the body torso to scan. This means that each Internal Sensor module had to have a preassigned parameter that stores their position. At this point, the external scanner focused on each scanning volume and sending an IRB, which consisted of just a single bit for simplicity. If the package reached the internal sensors in a certain position, then this will reply back with an ACK, which was made of one bit as well. In order for an information bit to reach its destination, the ultrasonic beam needed to have an intensity higher than the receiver's sensitivity. ADA's real-life simulation results are shown in FIGS. 20A-20D. Particular emphasis was put on the discovery time and the discovery energy when a full body torso scan is run for ten IMDs over the scanning accuracy (scanStep). Then the discovery probability was tested over the scanning range (ratio between the focused beam area and the IMD size) for different scanning granularity. In the end, a statistical analysis of the energy consumption over the number of nodes/IMDs was ran.

The results in FIG. 20A show the discovery time for ten IMDs while sweeping the scanning accuracy (scanRange). Here, the discovery time starts from 1500 ms when scanRange=5 cm (high accuracy) and going down to 100 ms when scanRange >11 cm (coarse accuracy). Similarly, the results in FIG. 20B show the discovery energy while sweeping the accuracy. As was expected, the energy goes down when less accuracy was required because there are fewer regions to scan (less mesh points). For example, in order to minimize the discovery time and the discovery energy, the optimal scanDelta needs to be found. The accuracy sets the minimum delta between two adjacent scanning regions: if the delta is smaller than the device size, this can be reached by the packaged signal. Another parameter can be defined, scanRange, as the ratio between the focused area with the phased array technique and the IMD size. At this point, the accuracy would depend on the IMD size: the smaller the implanted device size, the higher is the accuracy would be required to find it. In FIG. 20C are shown the number of discovered nodes (percentage relative to the total ten IMDs) as a function of the scanRange and scanDelta (shown by the shades of the columns in the graph). As the scanRange approached 100%, all the nodes were discovered. Similarly, for a given fixed scanRange, the number of discovered nodes increased with decrease of scanDelta (improved accuracy). This allowed the conclusion that the optimal scenario is then the focused region of the phased array matches the dimension of the smallest IMD, which puts a limit to the optimal discovery time and energy. The graph in FIG. 20D shows the distribution of the energy consumption while changing the number of the nodes inside the body torso. Furthermore, for each number of IMDs, a hundred different simulations were run with different random distribution of their relative positions. This allowed to get a more significant statistical distribution. From FIG. 20D, the energy consumption ranges from 2.6 down to 0.2 mJ depending on the number of nodes and the scanning step.

In this work, the first ADA for INs was successfully demonstrated by exploiting the phased array capability of pMUT chips. ADA was implemented in a discrete event IN simulator based on experimental results. ADA shows very good real-time (RT) capabilities, with a full scanning time down to 100 ms and energy consumption down to 0.2 mJ, for a body torso of 60×30×20 cm³. This can help medical providers with long-term diagnoses of chronic disease which require continues monitoring and drug adjustments, while being noninvasive.

### Example 5: Dual Range and High Data-Rate Intrabody Communication Transceiver Based on pMUTs

In this work, the implementation of a dual distance range (short d_{S}=3.5 cm and long d_{L}=13.5 cm, distance applications) and high bandwidth, high data-rate transceiver (BW 200 kHz and Data-Rate 400 kbits/s) for intrabody communication links based on pMUTs was demonstrated. The transceiver included a Quadrature Phase-Shift Keying (QPSK) modulation and demodulation scheme implemented in a Universal Software Radio Peripheral (USRP). The intrabody antennas (transceiver and receiver) each included a 10×10 uni-morph pMUT array (FIG. 23) based on aluminum nitride (AlN) with circular shape design and resonance frequency f≈1700 kHz. The arrays were embedded in a tissue phantom to mimic human tissue properties and were coupled with ultrasound gel to avoid air gaps. The system was tested by serializing a 100×50 pixels optical image in a sample bit stream and transmitting it over the intrabody acoustic link. The sampling rate was set at twice the bandwidth of the pMUTs (based on the Nyquist theorem). The detected BER was 1E-4 and 1E-1 for short and long range, respectively, demonstrating the functionality of the pMUT intrabody transceiver. These levels of BER allowed for perfect reconstruction of the original data by time-averaging successive frames.

Two pMUT arrays were fabricated using the example process shown in FIG. 8. An optical image of one pMUT array is shown in FIG. 23, with scale bar 200 μm. The individual elements were tested for displacement sensitivity with a laser vibrometer resulting in S_{disp}=10 nm/V. The array directivity was mainly influenced by the ultrasonic wavelength λ=c/f and the array pitch set at pitch=λ/10, which was further examined in the MATLAB simulation shown in FIG. 24. Given the resonance frequency of f≈700 kHz and sound velocity in the human tissue of c=1500 m/s, it was shown that λ=2.1 mm and pitch=210 μm. This configuration made the array an omni-directional radiating element with an aperture=104° (or side blind angle of α=38°) as depicted in FIG. 24.

The transmission sensitivity of the array was computed, which is the SPL at a certain distance given an input signal of 1 V. This resulted in S_{TX}=144 dB/V at 1 m from the array (standard commercial measurement distance) and S_{TX}=161 dB/V at 13.5 cm (the location of a receiving array in this experiment). Similarly, the receiving sensitivity of the array was evaluated, which is the received voltage (in dBV, V_{ref}=1 V) when applying a reference input pressure level of 1 Pa (SPL=120 dB and P_{ref}=1 μPa in water or tissue), resulting in S_{RX}=−78 dBV.

A raw optical image of 100×50 pixels was serialized in MATLAB to create a bit stream for the communication scheme (FIG. 25A). FIG. 25A depicts the raw optical image at left and the bit string combined into raw data. Each pixel consists of a RGB vector of 3 integers (0-255) that can be converted into an 8-bit string, for a total of 24 bits for the vector. At this point all the pixels were concatenated in a bit stream resulting in a total raw data of Data_{RAW}=120 kbits, which is shown at the right of FIG. 25A.

Secondly, the bit stream was encoded with a QPSK modulation, which allows to encode 2 bits per second (FIG. 25B). The modulation was done asynchronously, eliminating the need for a clock. On the other hand, there was the need to add overhead information to the raw data in order for the receiver to detect it. This increase in data length is of approximately 10%, resulting in Data_{QPSK}=132 kbits.

Finally, the QPSK data was up converted by the USRP at the operation frequency of the pMUT array and transmitted through a tissue phantom that mimics the human tissue properties (FIG. 25C). For example, each of the two pMUTs can be configured to transmit and to receive depending on the connected configuration. FIG. 25C depicts the transmitting pMUT array at left of the tissue phantom and the receiving pMUT array at right of the tissue phantom. The pMUT array bandwidth had been previously measured and equals to BW≈200 kHz translating in a Data-Rate≈400 kbits/s. On the receiving side of the intrabody ultrasonic transmission link, the signal is down converted to baseband by another USRP and sampled at twice the bandwidth for perfect reconstruction (Nyquist theorem). The signal required constant frequency and frame synchronization. At this point the data bit stream was demodulated from the QPSK scheme and re-assembled in an RGB pixels matrix.

The functionality of the transceiver was tested both for short range d_{S}=3.5 cm (FIG. 26A) and long range d_{L}=13.5 cm (FIG. 26B) applications. Once the data was reconstructed into an image, this was compared to the original image in order to compute the BER, which corresponds to the percentage of corrupted pixels by the ultrasonic channel. Similarly, the base-band spectrum of the received signal was acquired in order to estimate the SNR, which is shown at the right of FIG. 26A and FIG. 26B. The experiment showed a BER_{S}≈1E-4 with an SNR_{S}≈35 dB and a BER_{L}≈1E-1 with an SNR_{L}≈15 dB respectively for short and long distance.

The implementation of a QPSK ultrasonic transceiver for intrabody communication links using pMUT array as radiating elements was accomplished, supporting both short and long range up to 13.5 cm. The long distance can allow reaching most of the IMDs, such as a pacemaker implanted at about 12 cm. The achieved levels of BER allow perfect reconstruction of the original data through time averaging of successive frames. Using an image as transmitted data allowed a direct visual interpretation of the BER and the quality of the ultrasonic channel.

### REFERENCES

N. Songhvi, K. Hynynen, and F. Lizzi, “New developments in therapeutic ultrasound,” IEEE engineering in medicine and biology magazine, vol. 15, no. 6, pp. 83-92, 1996.
J. E. Kennedy, “High-intensity focused ultrasound in the treatment of solid tumours,” Nature reviews cancer, vol. 5, no. 4, p. 321, 2005.
S. A. Quadri, M. Waqas, I. Khan, M. A. Khan, S. S. Suriya, M. Farooqui, and B. Fiani, “High-intensity focused ultrasound: past, present, and future in neurosurgery,” Neurosurgical focus, vol. 44, no. 2, p. E16, 2018.
J. R. McLaughlan, An investigation into the use of cavitation for the optimisation of high intensity focused ultrasound (HIFU) treatments. PhD thesis, Institute of Cancer Research (University Of London), 2008.
E. Demirors, G. Alba, G. E. Santagati, and T. Melodia, “High data rate ultrasonic communications for wireless intra-body networks,” in 2016 IEEE International Symposium on Local and Metropolitan Area Networks (LANMAN), pp. 1-6, IEEE, 2016.
F. V. Pop, B. Herrera, C. Cassella, G. Chen, E. Demirors, R. Guida, T. Melodia, and M. Rinaldi, “Novel pmut-based acoustic duplexer for underwater and intrabody communication,” in 2018 IEEE International Ultrasonics Symposium (IUS), pp. 1-4, IEEE, 2018.
Y. Lu, H. Tang, S. Fung, Q. Wang, J. Tsai, M. Daneman, B. Boser, and D. Horsley, “Ultrasonic fingerprint sensor using a piezoelectric micromachined ultrasonic transducer array integrated with complementary metal oxide semiconductor electronics,” Applied Physics Letters, vol. 106, no. 26, p. 263503, 2015.
X. Jiang, H.-Y. Tang, Y. Lu, E. J. Ng, J. M. Tsai, B. E. Boser, and D. A. Horsley, “Ultrasonic fingerprint sensor with transmit beamforming based on a pmut array bonded to cmos circuitry,” IEEE transactions on ultrasonics, ferroelectrics, and frequency control, vol. 64, no. 9, pp. 1401-1408, 2017.
Z. Zhou, S. Yoshida, and S. Tanaka, “Epitaxial pmnn-pzt/si mems ultrasonic rangefinder with 2 m range at 1 v drive,” Sensors and Actuators A: Physical, vol. 266, pp. 352-360, 2017.
F. V. Pop, B. Herrera, C. Cassella, and M. Rinaldi, “pmut-based real-time (rt) acoustic discovery architecture (ada) for intrabody networks (in),” in 2019 Joint Conference of the IEEE International Frequency Control Symposium and European Frequency and Time Forum (EFTF/IFC), pp. 1-2, IEEE, 2019.
B. Herrera, F. Pop, C. Cassella, and M. Rinaldi, “Aln pmut-based ultrasonic power transfer links for implantable electronics,” in 2019 20th International Conference on Solid-State Sensors, Actuators and Microsystems & Eurosensors XXXIII (TRANSDUCERS & EUROSENSORS XXXIII), pp. 861-864, IEEE, 2019.
N. G. Berg, T. Paskova, and A. Ivanisevic, “Tuning the biocompatibility of aluminum nitride,” Materials Letters, vol. 189, pp. 1-4, 2017.

1. A system for ultrasonically heating biological tissue, comprising:
a device implantable in a subject's body, the device comprising:
a substrate, and
an array of piezoelectric micromachined ultrasonic transducers (pMUTs) supported on the substrate; and
a controller operative to control the array to emit and focus ultrasound transmissions at biological tissue in the body to heat the biological tissue.

2. The system of claim 1, wherein each of said pMUTs comprises a layer of piezoelectric material sandwiched between two electrode layers, and wherein the substrate comprises an insulating layer between a base layer and one of the electrode layers.

3. The system of claim 2, wherein each of the piezoelectric material layer and the insulating layer has a thickness from about 200 nm to about 5000 nm.

4. The system of claim 2, wherein the base layer comprises silicon, the insulating layer comprises silicon dioxide, the electrode layers comprise gold or platinum, and the piezoelectric layer comprises aluminum nitride, scandium doped aluminum nitride, lithium niobate, or a combination thereof.

5. The system of claim 1, wherein each pMUT of the array is independently addressable by the controller, and wherein the controller is operative to determine a focal point of ultrasound transmissions from the array by providing a time delay to an AC voltage applied to each pMUT of the array.

6. The system of claim 1 comprising two or more of said arrays, wherein each array is in communication with the controller, which is operative to determine a common focal point of ultrasound transmissions from the two or more arrays.

7. The system of claim 1, wherein the controller comprises a plurality of directly modulated ultrasound transducer circuits, each circuit comprising an inductor, a capacitor, a voltage input, and a bipolar junction transistor, and each circuit controlling operation of a different one of said array of pMUTs.

8. The system of claim 7, further comprising a microprocessor in communication with the plurality of transducer circuits and operative to provide an input voltage to each of the transducer circuits.

9. The system of claim 1, wherein the controller is implantable, or wherein said implantable device comprises the controller.

10. The system of claim 1, wherein the pMUTs produce ultrasound transmissions at a frequency in the range from about 20 kHz to about 200 MHz.

11. The system of claim 1, wherein the array comprises from 1×1 pMUT to about 200×200 pMUTs.

12. The system of claim 1, wherein the system is capable, when implanted in a subject's body, of heating biological tissue of the subject from about 37° C. to at least about 41° C.

13. An implantable, wearable, or portable medical device comprising the system of claim 1.

14. A method of ultrasonically heating a biological tissue in a subject, the method comprising:
(a) implanting into the subject's body (i) a device comprising an array of pMUTs supported on a substrate, wherein the pMUTs of the array are in communication with a controller operative to control the pMUTs of the array to emit and focus ultrasound transmissions, or (ii) the system of claim 1; and
(b) causing one or more of the pMUTs of the array to emit an ultrasound transmission focused on the biological tissue, thereby heating the tissue.

15. The method of claim 14, wherein two or more of said arrays are implanted, each array in communication with the controller, which is operative to determine a common focal point of ultrasound transmissions from the two or more arrays.

16. The method of claim 14, wherein the biological tissue is heated to at least about 41° C.

17. The method of claim 16, wherein the biological tissue is heated to at least about 60° C.

18. The method of claim 14, wherein the heated biological tissue comprises cancer cells.

19. The method of claim 18, wherein the cancer cells are selected from the group consisting of neck cancer cells, brain cancer cells, thyroid cancer cells, breast cancer cells, prostate cancer cells, kidney cancer cells, endometrial cancer cells, pancreatic cancer cells, lung cancer cells, esophageal cancer cells, bladder cancer cells, rectal cancer cells, cervical cancer cells, ovarian cancer cells, peritoneal cancer cells, sarcoma cancer cells, neuroblastoma cancer cells, leukemia cancer cells, melanoma cancer cells, and combinations thereof.

20. The method of claim 14, wherein the method is repeated one or more times, optionally with alteration of a focal point of the ultrasound transmissions.

21. The method of claim 14, wherein the method is combined with one or more of radiation therapy, immunotherapy, targeted drug therapy, chemotherapy, radiofrequency therapy, imaging, or hormone therapy.

22. The method of claim 14, wherein the method results in the death of cells of the biological tissue.
